(19) United States
(12) Patent Application Publication
(10) Pub. No.: US 2010/0131045 A1
(43) Pub. Date: May 27, 2010

(54) STENT DESIGNS, MATERIALS AND PROCESSING METHODS

(75) Inventors: Oren Globerman, Kfar-Shemaryahu (IL); Mordechay Beyar, Caesarea (IL)

Correspondence Address: MARTIN D. MOYNIHAN d/b/a PRTSI, INC., P.O. BOX 16446, ARLINGTON, VA, 22215, US

(73) Assignee: EXISTENT INC., Wilmington, DE (US)

(21) Appl. No.: 12/374,674

(22) PCT Filed: Jul. 24, 2007
(86) PCT No.: PCT/IL2007/000931

§ 371 Date: Dec. 14, 2009

Related U.S. Application Data
(60) Provisional application No. 60/832,589, filed on Jul. 24, 2006.

(51) Int. Cl. A61F 2/82

(52) U.S. Cl. 623/1.16; 623/1.15; 623/1.34; 72/367.1

(57) ABSTRACT

Stents and methods for design, usage and manufacture, using novel materials and designs, for example, high yield strength, low Young modulus, cold worked and/or with low elongation requirements.

### RELATED APPLICATIONS

The present application claims the benefit under 119(e) of U.S. Provisional Application No. 60/832,589 filed 24 Jul. 2006 the disclosure of which is incorporated herein by reference. This application is also related to U.S. Ser. No. 10/517,940, the disclosure of which is incorporated herein by reference.

### FIELD OF THE INVENTION

The present invention, in some embodiments thereof, relates to stent materials, stent designs and stent manufacture.

### BACKGROUND OF THE INVENTION

A main cause of death and disability in the modern world is the stenosis of blood vessels. Left unchecked, such stenosis can cause degeneration of tissues, such as the kidneys or the brain or even the acute failure of a tissue. For example the heart or the brain may experience an acute failure if such a stenosed vessel spasms or is clogged by floating debris (which debris is often fallout from a different stenosis).

A balloon expandable stent is typically a tiny tubular element designed to be inserted into a tubular vessel within the body, while in a first smaller diameter, and then plastically deformed to a second larger diameter, in order to support the vessel wall at the second diameter. Stents often cause a tissue reaction, which may cause lumen narrowing and obstruction (“restenosis”).

There is, apparently, a correlation between stent wall thickness and the amount of tissue reaction to the stent. (For more details, see “Intracoronary Stenting and Angiographic Results, Struts Thickness Effect on Restenosis Outcome” (ISAR-STEREO), Trial by A. Kastrati MD, J. Hehilli MD 2001; “In-Stent Restenosis in Small Coronary Arteries, Impact of Strut Thickness”, C. Briouri MD, PHD, C. Sarais, MD, J AM Coll Cardiology 2002). The disclosures of all articles, publications and patents mentioned in this application are incorporated herein by reference.

A typical metal coverage, of the vessel wall, when the stent is in an expanded state, is about 15%.

In addition a cell size (defined by surrounding struts) may affect the quality of support of vessel walls, for example, cells may be sized to prevent tissue prolapse into these cells

Exemplary stents are made of Cobalt-Chrome alloys, which may achieve yield strengths of up to 500-600 MPa once expanded within the body. These stents have higher yield strength than, for example, SS 316LVM stents (which has a yield strength of about 200 MPa).

As a consequence most coronary stents available in the market are characterized by strut thickness and width of 0.075 mm to 0.13 mm. Scheuermann (U.S. Pat. No. 7,101,391) teaches that a 70 to 80 μn thick 316L stainless steel offers sufficient strength to resist vessel forces.

Another limiting factor in stent design is the elongation properties of the material used. When in an annealed condition, the material typically expresses maximal elongation properties. For materials used in stent design, as the material is more cold-worked and less annealed, maximum elongation goes down. Passing the maximum elongation may cause a development of micro-cracks in the stent structures. For example, Stents formed of Stainless Steel 316 LVM alloy are supplied in a full annealed state (elongation of approximately 40%). This allows the stent to expand from its initial diameter (e.g., approximately 1 mm) to its final diameter (e.g., approximately 3-4 mm) without passing the maximal elongation properties (40%, in the case of annealed SS 316) at joints in the struts. Other materials having higher yield strengths than SS 316 typically do not have the elongation properties needed for the stent expansion (e.g., about 40%), and are therefore considered less useful for such expanding stents.

Another reason for preferring annealed materials over cold-worded materials is that cold-worked materials have a higher springback. For example, even an annealed stent, when expanded to a final diameter of 3 mm, will spring back immediately to about 2.7 mm, when released, independent of any radial forces applied by the surrounding blood vessel.

Another important factor that relates to the clinical performance of is the biocompatibility of the material used.

Some materials used in stents (e.g., metals that contain Nickel and/or Molybdenum) excrete small amounts of toxic materials, which may interfere biologically with the healing process of the tissue. A correlation was found between restenosis and a presence of such materials (for more details, see “Impact of Nickel Reduced Stainless Steel Implant on Striated Muscle Microcirculation: A Comparative In Vivo Study”, Clayton N. Kraft, Bjorn Burian 2001; “Relating Nickel-Induced Tissue Inflammation to Nickel Release In Vivo” by John C. Wataha, Norris L. O'Dell, 2001).

Attempts to introduce unfamiliar stent materials have been made in recent years, mainly with the intent to form a radio-opaque bio-compatible stent, such as Scheuermann's (U.S. Pat. No. 7,101,391) introduction of primary Niobium stent. Others have suggested the use of Tungsten-Rhenium conjointly with a second material, where the first contributes mostly to X-Ray compatibility and the second to its structural integrity (Mayer U.S. Pat. No. 5,628,787, Mayer PCT95/30384), or even a stent comprising essentially Tungsten-Rhenium alloy (Jansen, US application 20030181972) or Molybdenum-Rhenium Alloy (Xu, EP1539269B1). Generally, the designs suggested were standard stent designs.

Buergermeister et al. describe a design of high strength, controlled recoil stent (US2006/0020325), but their description encourages stress relieving the raw material during manufacturing, hence avoiding the use of a material in any cold-worked state.

The following references relate to properties of Tungsten-Rhenium:

Savitskii E. M. et al “PROPERTIES OF TUNGSTEN-RHENIUM ALLOYS” Metallovedenie j Term. Obrabotka HB No. 5489 Metallov, September, 1960, #9, pp. 20-25

Raffo Peter. L. “YIELDING AND FRACTURE IN TUNGSTEN AND TUNGSTEN-RHENIUM ALLOYS” NASA TN D-4567, Lewis Research center, May 1968.

Klopp W. D et al. “Mechanical properties of Dilute Tungsten Rhenium alloys” NASA TN D-3483, Lewis Research center, September 1969.

Matsuno et al. “Biocompatibility and osteogenesis of refractory metal implants, titanium, hafnium, niobium, tantalum and rhenium”, Biomaterials 22 (2001) pp. 1253-1262

Klopp et al. “Mechanical Properties of a Tungsten-23.4 percent Rhenium 0.27 percent hafnium-carbon alloy.” NASA TN D-6308, Lewis Research center, April 1971.

Bryskin “Rhenium and Rhenium alloys” The Minerals, Metals & Materials Society, 1997, pp. 262-265, 373-382, 647, 661-670, 717-727 and 806.

### SUMMARY OF THE INVENTION

A broad aspect of some embodiments of the invention relates to stents formed of high-strength materials and to designs that utilize the high-strength to provide various stent properties and to designs that compensate for one or more limitations of high strength materials.

An aspect of some embodiments of the invention relates to stents formed of cold worked materials. Exemplary materials include Stainless Steel, Cobalt-Chrome and refractory alloys based on Rhenium, specifically Tungsten-Rhenium, Molybdenum-Rhenium and Rhenium-Tungsten. Some additives may be provided as well.

In an exemplary embodiment of the invention, the materials are cold-worked to an extent that provides a desired tradeoff between increased strength and remaining elongation. For example, the materials may be cold worked to an extent that leaves at least 10%, 15% or 20% elongation. Optionally, the materials are cold worked to an extent that reduces the free elongation by 30%, 50%, 60% or smaller or intermediate amounts. By free elongation is meant the difference between the minimal elongation (completely cold worked) and the maximum elongation (annealed). In an exemplary embodiment of the invention, the desired tradeoff is determined by stent design and designed maximum expansion of the stent.

In one embodiment of the invention, the material used has a reduced Molybdenum and/or Nickel contents, while achieving high Yield strength of at least approximately 900-1000 MPa. In some embodiments and materials, to achieve this goal, the material has to be partially cold worked to have an elongation of between 15-25%.

In some exemplary embodiments of the invention, the material is an alloy. An example of an alloy is PP-1100, which, when it processed to a partially annealed state (of approximately 20% elongation) maintains a yield strength of about 900-1,000 MPa.

In an exemplary embodiment of the invention, the stent design compensates for an increased springback of cold-worked materials, by providing at least one stent section where an initial springback, even if relatively large, has a small (e.g., acceptable) effect on the springback of the stent as a whole. Optionally, the design includes struts that have a high intra-strut angle, at a joint deformed by stent expansion.

In an exemplary embodiment of the invention, reduced elongation is compensated for by using narrow struts (e.g., at least at joints), which are less sensitive to failure during bending, due to lower required elongation ability of the beam as compared to a same element having a same bending requirement but greater beam width.

An aspect of some embodiments of the invention relates to a stent with reduced footprint, the footprint comprising one or more of solid volume, strut thickness, strut width, strut cross-section, fin length, stent layer thickness on a balloon, moment of inertia along the stent radial axis when crimped) and/or circumferential density.

In an exemplary embodiment of the invention, a stent is provided with a low struts cross-section, which is less than half in width and thickness compared with common stent designs. The inventors believe these properties may reduce post PTCA restenosis rate (e.g., by reducing an amount of material and/or size of interfering elements) and/or improving implant's deliverability (e.g., by reducing a moment of bending of the strut).

It should be noted that stent's radio-opacity is optionally not factored into the stent design. Optionally, radio-opacity is provided by adding a radio-opaque marker to the stent or by forming as part of the stent to be specifically a radio-opaque marker. This being in spite of the stent, in some embodiments of the invention, being formed of materials that are considered to be radio-opaque (for normal-footprint stents).

In an exemplary embodiment of the invention, metal coverage ratio is reduced by using a lower strut width while optionally maintaining a cell area of 2-4 mm². In an exemplary embodiment of the invention, the stent has a metal coverage of less than 10%, less than 9%, less than 8% and optionally less than 5% or intermediate coverage. Optionally, when a different cell area and/or shape is desired, such smaller cells can be achieved while still remaining at low metal coverage, optionally below comparable stents, for example, 20%, 15%, 13% or less. Optionally, also strut thickness is reduced by 20%, 30, 40%, 50% or intermediate or greater values, which contributes to reduced total metal volume and/or area.

In an exemplary embodiment of the invention, deliverability is improved by providing struts, which, when in a folded (and generally axial) state (fins), are axially shorter. Optionally, the shortening is by at least 20%, 50% or 65%. Optionally, the fins are shorter than 0.9 mm, 0.75 mm, 0.6 mm, 0.5 mm, for example, for a 2.5 mm or 3.5 mm diameter stent. Optionally, at least part of the shortening is compensated for by increasing a number of folds (fins) in a circumferential direction. Optionally or alternatively, at least part is compensated for by increased intra-strut angle.

In an exemplary embodiment of the invention, delivery is improved by reducing the total diameter of the stent (e.g., the stent profile) when crimped on the delivery catheter's balloon, due to thickness reduction of the stent and/or increased ability to crimp stent.

In an exemplary embodiment of the invention, the stent is a strut-type stent including generally radial support elements and general axial support elements, optionally with cells between them of areas on the order of square mms. Similar strut sizes may be provided, by weaving, braiding or meshes.

In an exemplary embodiment of the invention, reduced strut width is used to allow a reduced elongation to support a same amount of bending or more, or allows increased bending at a joint to be provided for a same elongation.

An aspect of some embodiments of the invention relates to a stent with an increased number of fins in a circumferential direction. In one example, more than 26 fins are provided in at least one circumferential section, per 3 mm circumference (crimped). Optionally, the fins are defined as those strut elements which rotate substantially during deployment, for example, rotating more than 40 degrees. Optionally, the fins rotate from a substantially axial direction to a substantially circumferential direction.

In an exemplary embodiment of the invention, the increased number of fins is used to provide a desired circumference in the presence of a reduced fin length. Optionally or alternatively, the increased number of fins is used to provide smaller cell sizes. Optionally or alternatively, the increased number allows length reduction for improveing a stent's flexibility and conformability.

An aspect of some embodiments of the invention relates to a stent design including a higher intra-strut angle at joints which deform during expansion of the stent. In an exemplary embodiment of the invention, the angle is selected so that a springback of 0.3% to 0.8% causes a change in stent diameter of less than 8%, 5%, 4%, or intermediate values. In an exemplary embodiment of the invention, the angle is above 120 degrees, above 130 degrees, above 140 degrees, above 160 degrees, above 170 degrees or intermediate values. In an exemplary embodiment of the invention, the spring back causes such joints that interconnect strut sections to reduce in angle. However, by selecting a large angle, the effect on the stent diameter and length of the strut (e.g., linear length between the distal tips of the struts sections), is reduced.

In an exemplary embodiment of the invention, the cumulative effect of joint angle springback is approximated as a CR (circumference ratio), the ratio between a length of the strut along the strut contour and the linear length of the strut between two distal ends of the strut (e.g., where they connect to each other or other struts or stent longitudinal elements), when the strut is expanded to its maximum designed expansion.

In an exemplary embodiment of the invention, the CR is less than 1.5, for example, being less than 1.42, less than 1.4, less than 1.35, less than 1.3, less than 1.2, less than 1.1 or intermediate values.

In an exemplary embodiment of the invention, CR is reduced and joint angle (intra-strut angle) increased by providing a strut that is nearly straight (e.g., close to 180 degrees) when expanded to a maximum designed expansion.

In some cases, this large angle prevents the user from over-expanding a stent. While in some cases this may be useful, in other cases, the doctor has a desire to expand “a bit more”. Optionally, such additional expansion is provided using one or more joints (e.g., spiral sections) that open only at large forces.

In an exemplary embodiment of the invention, the stent as a whole provides an expansion of approximately 100%, 200%, 300%, 400% or lesser or higher or intermediate figures, from its initial diameter, while maintaining the deformation of the struts below the elongation limit of the material of which the stent is constructed.

Optionally, the material used is selected to be one with a high Young modulus and therefore a reduced spring back.

An aspect of some embodiments of the invention relates to using an alloy of Tungsten and Rhenium for manufacturing stent. In an exemplary embodiment of the invention, at least 70%, at least 80%, at least 90% or at least 95% of the stent volume is composed of such an alloy. In an exemplary embodiment of the invention, the alloy is at least partially cold-worked.

In an exemplary embodiment of the invention, the alloy includes at least 5% Rhenium and the balance is at least mostly Tungsten. In an exemplary embodiment of the invention, at least 20%, 25%, 26%, 27%, 28% or more of Rhenium is provided, optionally the amounts depending on complete solution of the Rhenium in Tungsten during manufacture.

In an exemplary embodiment of the invention, the alloy comprises a higher composition of Rhenium, for example, up to 40% or more. Optionally, a Rhenium/Tungsten alloy is used, in which the percentage of Tungsten is less than 20%, less than 10% or less, for example, as described in US patent publication 2005/238522. In an exemplary embodiment of the invention, an alloy with a high Young's modulus is selected (e.g., about 450 GPa). For this alloy (5% Tungsten), the elongation (when annealed) is 37% and Yield strength is 92 ksi (625 MPa). Optionally, this alloy is cold-worked as well, for example, 5%, 20%, 30% or intermediate or larger values.

In an exemplary embodiment of the invention, the alloy used includes additional components, for example, metal-carbides or metal-oxides are suggested. In some embodiments, these additives suppresses the growth of recrystalized grains during annealing, which can allow increased ductility, optionally by grain size being smaller than 10 μm, less than 8 μl , less than 6 μm. Exemplary additives include Zirconium-Carbide, Hafnium-Carbide, or Thorium-Oxide with up to 2% of the weight percentage.

In an exemplary embodiment of the invention, Osmium is added (e.g., up to 4.5%, and, for example, about 1.6-1.8%) to further increase the influence of Rhenium on the alloy's ductility/elongation to break.

An aspect of some embodiments of the invention relates to a stent formed of radio-opaque materials, but whose structure is not considered radio-opaque under normal imaging x-ray angiography conditions conditions. Optionally, the stent includes one or more specific radio-opaque regions in addition to the basic structure of the stent. Optionally, at least 90% of a surface of the stent is not radio-opaque. Optionally, the stent is formed of at least 50%, at least 70%, at least 90% radio-opaque materials. Optionally, the radio-opaque materials are at least 140%, 160%, 200% or more as radio-opaque as stainless steel 316. In an exemplary embodiment of the invention, the stent is formed of an alloy of Tungsten and/or Rhenium. In an exemplary embodiment of the invention, the stent is less radio-opaque than an expanded ARTHOS stent, by a factor of 1.2, 1.5, 2, 3, 4 or greater or intermediate numbers.

An aspect of some embodiments of the invention relates to a method of manufacturing a stent from cold-worked and/or high-strength (e.g., yield strength above 1000 MPa, above 1500 MPa, above 2000 MPa, above 2500 MPA or intermediate values) materials. In such materials, rolling for area reduction may require many repetitions, as each step may allow only 10%. In an exemplary embodiment of the invention, instead of rolling the tubing to the final dimension, the tubing's inner and outer diameters are machined from a tubing or a rod to their desired dimensions, for example by grinding, Electro-discharge or any other machining process.

In an exemplary embodiment of the invention, the stent is manufactured by forming a tube or a rod of the material, for example, by sintering, cold working the tube, machining the tube to size and cutting out parts of the tube, for example using laser cutting. Optionally, the tube is then polished, for example using electro-polishing. Optionally or alternatively, the tube may be spot annealed, for example, using an electron gun.

An aspect of some embodiments of the invention relates to a method of stent design in which an existing stent design is adapted for materials which have a higher yield-strength, lower Young's modulus and/or lower elongation.

In an exemplary embodiment of the invention, an existing stent design is provided and the material is changed to a material which has different properties, for example, being cold-worked and/or being a different composition of matter. Then, one or more of thickness, width, CR, number of fins and/or length of fins are modified. Optionally, the modification maintains, improves or does not degrade by more than 10% or 20%, diametric spring back of the stent. Optionally or alternatively, the change in material properties is used to meet a criteria, such as decreasing spring back effect.

In an exemplary embodiment of the invention, when the stent is used (e.g., placed on a balloon catheter and guided, optionally using a guide wire to a stenosed location), it is selected with the user being aware that there may be a reduced capacity for over inflation. Optionally, instructions packaged with the stent emphasize this point. Optionally, the stents are manufactured in runs of about, 10, 50, 100, 100 or greater or intermediate numbers.

There is thus provided in accordance with an exemplary embodiment of the invention, a stent formed of at least 60% of a material that is cold-worked to an extent of at least 5%.

There is also provided in accordance with an exemplary embodiment of the invention, a stent utilizing a plurality of radial struts to provide structural support to a blood vessel, wherein at least 60% of the radial struts from which the stent is constructed are formed of a material that is cold-worked to an extent of at least 5%.

Optionally, said extent is at least 20%.

Optionally or alternatively, said extent is at least 30%.

Optionally or alternatively, said extent is at least 40%.

In an exemplary embodiment of the invention, said stent has at least one radial strut, wherein when said stent is expanded to a maximal predetermined diameter, said radial strut has an elongation of less than 30% applied thereto. Optionally, said elongation is less than 20%. Optionally or alternatively, said elongation is less than 15%. Optionally or alternatively, said elongation is at least 10%

In an exemplary embodiment of the invention, said material is stainless steel.

In an exemplary embodiment of the invention, said material is an alloy comprising Tungsten and Rhenium at percentages above 5% each.

In an exemplary embodiment of the invention, said material is an alloy comprising Molybdenum and Rhenium at percentages above 5% each.

In an exemplary embodiment of the invention, said stent as a whole springs back radially less than 10% when expanded to a maximum design diameter thereof.

In an exemplary embodiment of the invention, said stent as a whole springs back radially less than 5% when expanded to a maximum design diameter thereof.

In an exemplary embodiment of the invention, said stent is a coronary stent.

In an exemplary embodiment of the invention, said stent is a cerebral stent.

In an exemplary embodiment of the invention, said stent has a maximum design diameter of less than 3.5 mm.

In an exemplary embodiment of the invention, said stent has a maximum design diameter of less than 2.5 mm.

In an exemplary embodiment of the invention, said stent has a maximum design diameter of less than 4.5 mm.

In an exemplary embodiment of the invention, said material is stainless steel, said stent is designed for a nominal expansion diameter of 2.5 mm or less and wherein said stent is formed mostly of weight bearing struts having a width of about or less than 0.05 mm.

In an exemplary embodiment of the invention, said material is stainless steel, said stent is designed for a nominal expansion diameter of 3.5 mm or less and wherein said stent is formed mostly of weight bearing struts having a width of about or less than 0.06 mm.

In an exemplary embodiment of the invention, said material is a tungsten-rhenium alloy, said stent is designed for a nominal expansion diameter of 2.5 mm or less and wherein said stent is formed mostly of weight bearing struts having a width of about or less than 0.035 mm.

In an exemplary embodiment of the invention, said material is a tungsten-rhenium alloy, said stent is designed for a nominal expansion diameter of 3.5 mm or less and wherein said stent is limited mostly of weight bearing struts having a width of about or less than 0.045 mm.

In an exemplary embodiment of the invention, said stent comprises a plurality of struts, and wherein at least 40% by length of said struts have a width of less than 0.05 mm. Optionally, said 40% of struts have a width of less than 0.04 mm. Optionally, said 40% of struts have a width of less than 0.03 mm.

In an exemplary embodiment of the invention, said stent has a maximum design diameter which is at least 200% a crimped diameter.

In an exemplary embodiment of the invention, said stent has a maximum design diameter which is at least 300% a crimped diameter.

In an exemplary embodiment of the invention, said stent has a maximum design diameter which is at least 400% a crimped diameter.

In an exemplary embodiment of the invention, said stent, at a maximum design diameter thereof, has a wall coverage of less than 10%.

In an exemplary embodiment of the invention, said stent, at a maximum design diameter thereof, has a wall coverage of less than 6%.

There is also provided in accordance with an exemplary embodiment of the invention, a stent defining a cylindrical surface and having a nominal maximum expansion diameter and having a ratio between said surface and a surface area of said stent of more than 1:10, at said diameter.

In an exemplary embodiment of the invention, said ratio is greater than 1:15. Optionally, said ratio is greater than 1:20. Optionally or alternatively, said diameter is less than 4 mm. Optionally, said diameter is less than 3 mm.

In an exemplary embodiment of the invention, said stent is a coronary stent.

In an exemplary embodiment of the invention, said stent is a cerebral stent.

In an exemplary embodiment of the invention, said stent is a peripheral vessel stent.

In an exemplary embodiment of the invention, said stent is a non-vascular stent.

There is also provided in accordance with an exemplary embodiment of the invention, a stent formed of a plurality of expandable radial struts which provide at least 60% of radial support to said stent, wherein a width of at least 40% of said struts is less than 60 microns.

In an exemplary embodiment of the invention, said struts have a thickness about equal to said width.

In an exemplary embodiment of the invention, the stent comprises an additional mesh of thin-diameter material.

In an exemplary embodiment of the invention, said struts have a width of at least 10 microns.

In an exemplary embodiment of the invention, said struts have a width of at least 15 microns.

In an exemplary embodiment of the invention, said struts have a width of less than 50 microns.

In an exemplary embodiment of the invention, said struts have a width of less than 37 microns.

In an exemplary embodiment of the invention, said struts have a width of less than 30 microns.

In an exemplary embodiment of the invention, said struts have a width of less than 20 microns.

In an exemplary embodiment of the invention, said stent provides vascular support at least matching a gold standard of support. Optionally, said struts are less than 60% the cross-section of struts of a same material in annealed condition, needed for providing said standard for said stent design. Optionally or alternatively, said struts are less than 60% the cross-section of struts of stainless steel 316LS, needed for providing said standard for said stent design. Optionally or alternatively, said cross-section is less than 40% of said cross section needed for SS. Optionally, said cross-section is less than 30% of said cross section needed for SS. Optionally, said cross-section is less than 20% of said cross section needed for SS.

There is also provided in accordance with an exemplary embodiment of the invention, a stent formed of a plurality of folded radial struts, said struts in folded condition defining fins, at least 80% of said fins having an axial length along the stent axis of less than ¼ of a nominal diameter of said stent.

Optionally, said length is less than ⅕ said diameter. Optionally, said length is less than ⅙ said diameter. Optionally, said length is less than 1/7 said diameter.

In an exemplary embodiment of the invention, said length is correct for at least 90% of said fins.

There is also provided in accordance with an exemplary embodiment of the invention, a stent having a designed maximum diameter of less than 3 mm, formed of a plurality of folded radial struts, each folded section being a fin, and having more than 26 fins in a circumferential direction for at least one axial section of the stent.

In an exemplary embodiment of the invention, the stent has more than 30 fins. Alternatively or additionally, the stent has more than 35 fins. Alternatively or additionally, the stent has more than 40 fins. Alternatively or additionally, the stent has more than 45 fins.

In an exemplary embodiment of the invention, the stent has at least three such axial sections which do not overlap.

There is also provided in accordance with an exemplary embodiment of the invention, a stent formed of a plurality of folded radial struts, including expansion joints, at least 30% of said joints being designed to straighten to an intra-strut angle of greater than 120 degrees.

In an exemplary embodiment of the invention, said angle is greater than 130 degrees. Optionally, said angle is greater than 150 degrees. Optionally, said percentage is greater than 50%.

In an exemplary embodiment of the invention, said percentage is greater than 70%.

There is also provided in accordance with an exemplary embodiment of the invention, a stent formed of a plurality of folded radial struts, at least 40% of said struts being designed to straighten so that a ratio between a physical length of the strut and a linear length between ends of the strut is less than 1.4.

Optionally, said ratio is less than 1.26.

In an exemplary embodiment of the invention, said struts effect an elongation of about or less than 20% when straightening out to a maximum design diameter.

In an exemplary embodiment of the invention, said at least 40% of struts comprise at least 60%. Optionally, said at least 40% of struts comprise at least 80%.

In an exemplary embodiment of the invention, said stent is an arterial stent with a final diameter 3.5 mm or less.

There is also provided in accordance with an exemplary embodiment of the invention, a stent formed of an alloy including Tungsten and Rhenium, wherein a percentage of Rhenium is greater than about 25%.

Optionally, said percentage is greater than about 26%. Optionally, said Rhenium is in a percentage of greater than 50%. Optionally, said Rhenium is in a percentage of greater than 80%.

In an exemplary embodiment of the invention, said alloy includes up to 5% of one or more additives selected form the group of Osmium, Carbides and Oxides.

There is also provided in accordance with an exemplary embodiment of the invention, a stent comprising a structure, said structure being formed of at least 80% by volume of a radio-opaque material more opaque than stainless steel 316, said structure being less radio-opaque than by a factor of at least 1.4 than a stent of same geometric design formed of stainless steel 316 with strut cross-sections suitable for a stainless steel design.

Optionally, said stent includes at least one radio-opaque marker.

In an exemplary embodiment of the invention, said factor is at least 2.

There is also provided in accordance with an exemplary embodiment of the invention, a method of manufacturing a stent, comprising:

forming a tube of a metal or an alloy thereof;

cold working the tube; and

cutting said tube to form said stent.

Optionally, the method comprises machining said tube to size.

There is also provided in accordance with an exemplary embodiment of the invention, a method of stent design, comprising:

(a) providing a stent design including a plurality of struts;

(b) modifying a material property of said design;

(c) reducing a strut width for at least 40% of said struts; and

(d) modifying at least one of intra-strut angle, strut CR, number of fins and length of fins in a manner which maintains a diametric recoil in a same nominal diameter at less than 5%, and maintains a same minimal required radial resistance

Optionally, said (b) comprises selecting a degree of annealing.

Optionally, said (b) comprises selecting a degree area reduction each of the tubing production phases.

Optionally, said (b) and said (d) are repeated iteratively.

Optionally, the method comprises selecting a material property such that elongation of said material is consistent with the geometric design of said stent.

There is also provided in accordance with an exemplary embodiment of the invention, a stent manufactured according to specification generated by the methods as described herein.

### BRIEF DESCRIPTION OF THE FIGURES

In the following drawings, identical structures, elements or parts that appear in more than one drawing are generally labeled with the same numeral in all the drawing in which they appear. Dimensions of components and features shown in the drawings are chosen for convenience and clarity of presentation and are not necessarily shown to scale. The drawings are listed below.

FIG. 1 is perspective showing of a stent;

FIG. 2 is a showing of relative cross-sections of struts of stents in accordance with prior art and in accordance with exemplary embodiments of the invention;

FIGS. 3A and 3B illustrate the opening of a strut in a stent having a low intra-strut angle;

FIGS. 4A and 4B illustrate the opening of a strut in a stent having a high intra-strut angle;

FIGS. 5A and 5B illustrate the opening of strut having a greater number of fins, in accordance with an exemplary embodiment of the invention;

FIG. 6A is a graph showing increased torque resistance and springback in a cold-worked material as compared to an annealed material, normalized to a same toque resistance, thereby reducing the width of the cold-worked;

FIG. 6B is a stress-strain diagram of some materials which may be used for manufacturing stents, in accordance with exemplary embodiments of the invention;

FIG. 7 is a graph illustrating how a low intra-strut angle can compensate for increased springback, in accordance with exemplary embodiments of the invention; and

FIGS. 8-10 are charts showing how a stent design may be optimized, in accordance with exemplary embodiments of the invention.

### DETAILED DESCRIPTION OF EXEMPLARY EMBODIMENTS OF THE INVENTION

### Overview

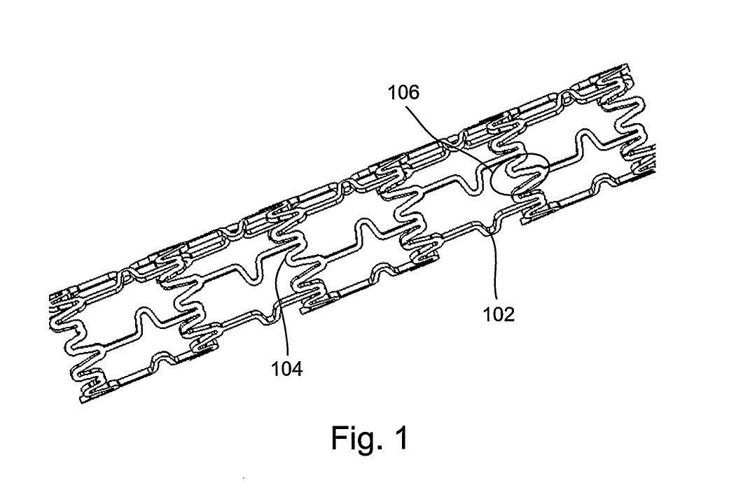

FIG. 1 is a schematic showing of a stent 100, which comprises a plurality of radial struts 102 and a plurality of longitudinal struts 104. In the particular example shown, the stent comprises a plurality of cylindrical segments interconnected by longitudinal struts. The longitudinal struts connect the radial strut along the longitudinal axis and/or participate in vessel wall scaffolding.

Each cylindrical segment is composed of one or more circumferentially arranged radial struts which carry most of the load imposed on the stent, which load is generally the vessel inward collapse forces.

It should be noted that various embodiments of the present invention apply to other stent designs as well, for example, to stents formed of a mesh with elements that are general all both axial and circumferential in orientation. Optionally, the design may be similar to or use features (mechanical and otherwise) that of one or more of the following stents, as well as other stents known in the art:

| Coronary stent | Manufacturer | Stent Design | Strut design |
|---|---|---|---|
| AMG ARTHOS | Amg GmbH, | Laser-cut slotted | Connected loops |
| CORONARY STENT | Raesfeld-Erle, | tube | |
| IMPLANTATION | Germany | | |
| AMG ARTHOS INERT | Amg GmbH, | Laser-cut slotted | Connected loops |
| CORONARY STENT | Raesfeld-Erle, | tube | |
| IMPLANTATION | Germany | | |
| ANTARES STARFLEX | InFlow | Homogeneous, | Oval strut cross-section |
| CORONARY STENT | Dynamics AG, | multicellular stent | |
| SYSTEM | Munich, | structure with | |
| | Germany | alternating stiff | |
| | | and flex segments | |
| | | for longitudinal | |
| | | flexibility. | |
| AST LSK-VIEW STENT | Advanced | Serpentine | ‘C’ and ‘S’ |
| AND DELIVERY | Stent | w/Expandable | |
| SYSTEM | Technologies, | Side Hole | |
| | Pleasanton, | | |
| | CA, USA | | |
| BESTENT TM2 STENT | Medtronic | Laser-cut tube | Rectangular |
| | AVE, Santa | with solid gold | |
| | Rosa, USA | radiopaque end | |
| | | marker and no | |
| | | welding points. | |
| | | Characterized by | |
| | | flexible ‘S’ | |
| | | crowns and | |
| | | longitudinal ‘V’ | |
| | | crowns that cross | |
| | | each other in a | |
| | | junction which | |
| | | undergoes rotation | |
| | | during expansion. | |
| BIODIVYSIO AS | Biocompatibles | Interlocking | 0.002-0.003 inch (0.05-0.08 mm) |
| (ADDED SUPPORT) | Ltd, Farnham, | arrowheads with | wide and 0.004 |
| STENT RANGE WITH | Surrey, UK | rectangular, | inch (0.09 mm) thick |
| PC TECHNOLOGY TM | | rounded edge | |
| | | struts | |
| BIODIVYSIO OC | Biocompatibles | Interlocking | 0.002-0.003 inch (0.05-0.08 mm) |
| (OPEN CELL) STENT | Ltd, Farnham, | arrowheads with | wide and 0.004 |
| RANGE WITH PC | Surrey, UK | rectangular, | inch (0.09 mm) thick |
| TECHNOLOGY TM | | rounded edge | |
| | | struts | |
| BIODIVYSIO SV | Biocompatibles | Interlocking | 0.001-0.002 inch (0.03-0.05 mm) |
| (SMALL VESSEL) | Ltd, Farnham, | arrowheads with | wide and 0.002 |
| STENT RANGE WITH | Surrey, UK | rectangular, | inch (0.06 mm) thick |
| PC TECHNOLOGY TM | | rounded edge | |
| | | struts | |
| BX VELOCITY TM | Cordis, a | struts make of | Struts make of curved |
| STENT | Johnson & | curved sections | sections joining negative |
| | Jonson | joining negative | angle diagonals |
| | Company, | angle diagonals | interconnected with ‘N’ |
| | Warren, NJ, | interconnected | shaped flex segments. |
| | USA | with ‘N’ shaped | 0.0055 inch wall thickness |
| | | flex segments | 0.0052 inch strut width |
| THE CARBOSTENT | Sorin | Laser | — |
| SURIUS STENT | Biomedica | micromachined | |
| | S.p.A, Saluggia | tube, multicellular | |
| | (VC), Italy | architecture with | |
| | | curved segment of | |
| | | variable cross- | |
| | | section. | |
| THE CORDYNAMIC | Iberhospitex | Slotted tube stent | Sinus curve, rounded |
| APOLO STENT | SA, Barcelona, | | edges |
| | Spain | | |
| COROFLEX AND | B Braun | Laser-cut, slotted- | Electropolished, ellipto- |
| COROFLEX DELTA | Mersungen | tube with | rectangular, rounded- |
| CORONARY STENT | AG, Berlin, | sinusoidal ring | edges. |
| SYSTEMS | Germany | elements joined at | |
| | | their mid-points | |
| | | by flexible bridges | |
| DURAFLEX TM | Avantec | Circumferential | Dimensions: 0.005 inch |
| coronary stent system | Vascular | rings, linked with | thickness; 0.0042 inch |
| | Corporation, | flexible cross- | width |
| | San Jose, CA, | bridges | |
| | USA | | |
| EXPRESS CORONARY | Boston | Laser cut from a | Dimensions: |
| STENT SYSTEM | Scientific Inc, | stainless steel tube | 0.0052 inch thick; |
| | Natick, MA, | in a corrugate ring | 0.0024/0.0036 inch wide |
| | USA | pattern of Macro | |
| | | and Micro | |
| | | elements. Two | |
| | | different elements | |
| | | combined in one | |
| | | single design, | |
| | | offering a balance | |
| | | between | |
| | | flexibility and | |
| | | scaffolding, while | |
| | | addressing a host | |
| | | of other | |
| | | performance | |
| | | attributes. | |
| GENIC DYLIN STENT | Blue Medical | Helical sinusoidal | Rectangular with rounded |
| | Devices BV, | waves | edges. |
| | Helmond, The | | |
| | Netherlands. | | |
| GENIC SV STENT | Blue Medical | Helical sinusoidal | Rectangular with rounded |
| SYSTEMS | Devices BV, | waves | edges. |
| | Helmond, The | | Dimensions: |
| | Netherlands | | 0.11-0.12 mm wide; |
| | | | 0.10 mm thickness |
| GENIC LV STENT | Blue Medical | Helical sinusoidal | Rectangular with rounded |
| SYSTEMS | Devices BV, | waves | edges. |
| | Helmond, The | | Dimensions: |
| | Netherlands | | 0.10-0.13 mm wide; |
| | | | 0.11 mm thickness |
| GENIUS CORONARY | EuroCor | Tubular smooth | 0.004 inch (0.11 mm) |
| STENT | GmbH, Bonn, | rounded | thick |
| | Germany | | |
| IGAKI-TAMAI STENT | Igaki Medical | Zigzag helical coil | 0.007 inch (0.17) thick |
| | Planning, | | |
| | Kyoto Japan | | |
| JOSTENT CORONARY | Jomed AB, | Laser-cut from a | Rounded edges |
| STENT RANGE | Helsingborg, | stainless steel tube | |
| | Sweden | with no welding | |
| | | points, flexibility | |
| | | through spiral | |
| | | links with high | |
| | | radial strength. | |
| LUNAR CORONARY | InFlow | Homogeneous, | Oval strut cross-section |
| STENT SYSTEM | Dynamics AG, | multicellular stent | |
| | Munich, | structure, | |
| | Germany | ‘Starflex’ design | |
| MANEO STENT | DEVON | Multicellular | Altering round and oval. |
| | Medical, | slotted tube, | |
| | Hamburg, | special round edge | |
| | Germany | polishing | |
| MED-X FLEXY STENT | Med-Xcor, | Zigzag rings with | Rectangular with polished |
| | Trevoux, | few articulation | edge |
| | France | points | |
| THE MEDTRONIC AVE | Medtronic | Modular, | Ellipto-rectangular |
| MODULAR STENTS- | AVE, Inc. | sinusoidal ring, 10 | electropolished |
| S7 | Santa Rosa, | crown element | |
| | CA, USA | design | |
| MEDTRONIC AVE | Medtronic | Modular, | Ellipto-rectangular |
| MODULAR STENTS- | AVE, Inc. | sinusoidal ring, 6 | electropolished |
| S660 | Santa Rosa, | crown element | |
| | CA, USA | design | |
| MEGAFLEX | EuroCor | Tubular, smooth | 0.004 inch (0.11 mm) |
| XORONARY STENT | GmbH, Bonn, | rounded | thick |
| | Germany | | |
| MULTI LINK PENTA | Guidant | Laser cut from a | Rounded struts with VTS |
| CORONARY STENT | Vascular | stainless steel | technology: allowing the |
| SYSTEM | Intervention | tube. Pattern | struts to be thin where |
| | Group, Santa | based on the | flexibility is needed and |
| | Clara, CA, | traditional | thick in other areas to |
| | USA | Guidant Multi- | provide radiocapacity |
| | | Link, corrugated | (VTS = variable thickness |
| | | ring footprint. | strut) |
| | | Each ring is | |
| | | connected by 3 | |
| | | links (3-3-3 | |
| | | design) that have | |
| | | 5 turns embedded | |
| | | within the ring. | |
| MULTI LINK RX | Guidant | Laser cut from a | Unsupported surface area |
| PIXEL CORONARY | Vascular | solid stainless | 2.0 mm2 and 4.0 mm2 at |
| STENT SYSTEM | Intervention | steel tube in the | 2.5 mm |
| | Group, Santa | corrugated ring | |
| | Clara, CA, | pattern. Each ring | |
| | USA | is connected by 3- | |
| | | 3-3 links. | |
| MULTI LINK RX | Guidant | Laser cut from a | Proprietary. Unsupported |
| ULTRA CORONARY | Vascular | solid stainless | surface area 3.8 mm2 AT |
| STENT SYSTEM | Intervention | steel tube in the | 3.5 mm. |
| | Group, Santa | corrugated ring | |
| | Clara, CA, | pattern. Each ring | |
| | USA | is connected by 3- | |
| | | 3-3 links. | |
| NEXUS AND NEXUS II | Occam | Laser cut from a | 0.0047 inch (0.120 mm) |
| CORONARY STENT | International | stainless steel tube | thick |
| | BV, | | 0.0041 inch (0.105 mm) |
| | Eindhoven, | | wide |
| | The | | |
| | Netherlands | | |
| NIR AND NIRFLEX | Medinol Ltd, | | Square, transform from |
| CORONARY STENTS | Israel | | flexible to rigid. |
| | | | NIROYAL: Rounded |
| | | | square, transforms from |
| | | | flexible to rigid upon |
| | | | expansion. |
| PROLINK STENT | Vascular | Laser cut from | 0.0028 mm wall, 0.0028 mm |
| | Concepts | stainless steel tube | strut width |
| | Limited, | in a ring pattern, | |
| | Crawley, UK | ring | |
| | | interconnected by | |
| | | three alternating | |
| | | links. | |
| PROPASS STENT | Vascular | Laser cut from | 0.0028 mm wall, 0.0028 mm |
| | Concepts | stainless steel tube | strut width |
| | Limited, | in a ring pattern, | |
| | Crawley, UK | ring | |
| | | interconnected by | |
| | | three alternating | |
| | | links. | |
| CORONARY R STENT | Orbus Medical | Dual helical | Electropolished |
| | Technologies, | slotted tube | rectangular cross-section |
| | Inc, Fort | featuring three | with rounded edges. |
| | Lauderdale, FL, | dimensional | |
| | USA | conformability | |
| | | and squared end- | |
| | | zones. | |
| RITHRON AND | Biotronik | Tubular slotted | |
| RITHRON-XR | GmbH, Berlin | | |
| CORONARY STENTS | Germany | | |
| SEAQUEST STENT | CathNet- | Slotted tube. | |
| | Science SA, | | |
| | Paris, France | | |
| SPIRAL FORCE STENT | Bolton Medical | Spiral cut with C- | Straight struts |
| | Inc. Fair Lawn, | joints | |
| | NJ, USA | | |
| TSUNAMI CORONARY | Terumo | Several radial | Square |
| STENT SYSTEM | Corporation, | diamonds joined | |
| | Japan | by double | |
| | | connector | |
| ZEBRA STENT | Bolton Medical | Spiral cut with C- | Straight struts |
| | Inc. Fair Lawn, | joints | |
| | NJ, USA | | |
| BOSTON SCIENTIFIC | Boston | | |
| ANTIPROLIFERATIVE, | Scientific | | |
| PACLITAXEL | Corporation | | |
| ELUTING STENTS | Inc | | |
| (TAXUS) | | | |
| MULTI-LINK TETRA | Guidant | Laser cut from a | Unsupported surface area |
| DTM DRUG-ELUTING | Vascular | solid stainless | 3.4 mm2 at 3.05 mm |
| CORONARY STENT | Intervention | steel tube in the | |
| SYSTEM | Group, Santa | corrugated ring | |
| | Clara, CA, | pattern. Each ring | |
| | USA | is connected by 3- | |
| | | 3-3 links. | |
| PHYTIS DLC | Phytis Medical | Slotted tube with | |
| (DIAMOND-LIKE | Devices, | rounded edges and | |
| CARBON) COATED | GmbH, Berlin, | multiple curved | |
| STENTS | Germany | links | |
| PHYTIS DOUBLE- | Phytis Medical | | |
| COATED STENT | Devices, | | |
| | GmbH, Berlin, | | |
| | Germany | | |
| QUANAM QUADS-QP2 | Boston | | |
| STENT | Scientific | | |
| | Corporation | | |
| | Inc/Quanam | | |
| | Medical, Santa | | |
| | Clara, CA, | | |
| | USA | | |
| SUROLIMUS-ELUTING | | | |
| BX VELOCITY STENT | | | |

Designs of these stents are shown in “Handbook of Coronary Stents” 4^{th} edition, edited by P W Serruys and B J Rensing, published by Martin Dunitz, UK (2002) ISBN 1-84184-093-9, the disclosure of which is incorporated herein by reference.

As used herein, radial strut elements are parts of struts which rotate towards a more circumferential orientation during stent expansion, for example, by an angle of 30, 40, 50, 60, 70, or 80 degrees or intermediate amounts.

In accordance with exemplary embodiments of the invention, it is desired to reduce the metal coverage of a stent (or provide a stent with reduced metal coverage). In accordance with other embodiments, additional strength or other properties are provided, while maintaining low and/or otherwise acceptable coverage levels.

### Narrow Strut

In an exemplary embodiment of the invention, reduced coverage is provided by using narrower struts. Optionally, the struts are also made thinner. Optionally, the struts remain thicker than their width.

In an exemplary embodiment of the invention, struts are made thinner by making them stronger, for example, by cold-working and/or by selecting suitable materials. It is noted that even when some of the materials were suggested in the art, for a same application, struts as narrow as described herein were apparently not conceived of.

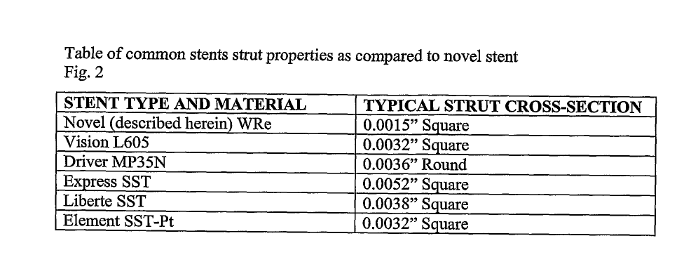

FIG. 2 is a showing of relative cross-sections of struts of stents in accordance with prior art and in accordance with exemplary embodiments of the invention. In one embodiment of the present invention, the stent has a strut thickness and width of less than 50% than other available stents for the same application. For example, in the coronary stent case, it is desired a strut thickness (204) and width (202) between 20 μm and 60 μm, and optionally less than 50μ are achieved (First strut from the left on FIG. 2). For example, for a cerebral artery stent, strut dimensions may be, for example, 30 μm 20 μm, 15 μm, 10 μm or smaller or intermediate or greater sizes.

When it is assumed that stent strength is measured primarily (or at least in a dominant manner) by its ability to resist torque imposed on its radial struts (after they are plastically deformed) by the blood vessel, the strut parameters may be modeled by a torque-curvature relationship for an elastic-ideally-plastically rectangular hinge (with a cross strut thickness of ‘d’ and a width of ‘b’).

The torque and the resulting beam curvature are described by the equation for the plastic zone (K>KE):

$$M = \frac{3}{2} {M}_{E} * [ 1 - \frac{1}{3} {( \frac{{K}_{E}}{K} )}^{2} ] ]$$

### Where:

a) M_{E}—is the yield torque (i.e. the required torque to begin plastic deformation), and is defined for a rectangular beam by

$$\frac{b * {d}^{2}}{6} * {σ}_{yield}$$

b) K is the curvature of the strut and defined by

$$K = \frac{1}{R} = \frac{2 * ɛ}{d}$$

c) K_{E}—is the yield curvature of the strut defined by

$${K}_{E} = \frac{1}{R} = \frac{2 * {σ}_{yield}}{E * d}$$

For the Elastic zone (K<KE) the torque increment is linear with K.

As can be seen, the torque is primarily affected by the M_{E} Component, with a milder effect of the K_{E}. These relations show that given higher material yield strength a same torque resistance could be achieved by a thinner strut.

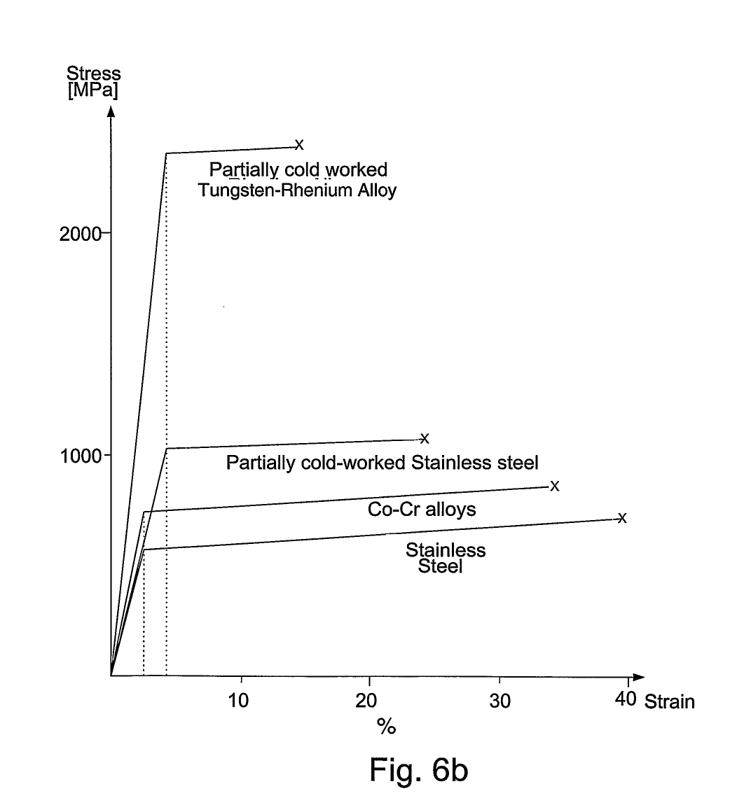

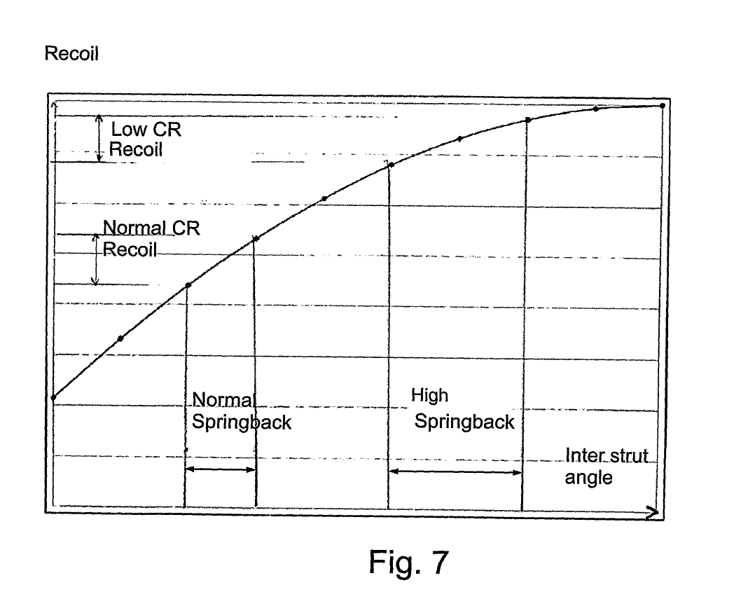

FIG. 6A is a graph showing increased torque resistance and spring back in a cold-worked material as compared to an annealed material

FIG. 6A also shows relative M vs. K for a material in annealed and cold-worked states (=higher yield strength), shows that M increases more slowly for a same amount of material.

After achieving maximal torque resistance, under relatively mild strains, it is illustrated in FIG. 6A that imparting additional curvature in the plastic zone cannot further increase the ratio of plastic curvature. However, the ratio of the elastic to plastic curvature is reduced. In some cases, additional curvature or larger expansion of the struts may reduce percentage of recoil.

In an exemplary embodiment of the invention, struts are selected to be of a size and/or geometry that enhances fast coverage by neo-intima. Optionally, the struts are small enough to be covered at least twice as fast, optionally 3 times as fast as comparable bare-strut stents (stents without an intra-strut covering of polymer or metal mesh (e.g., as described using ion assisted e-beam evaporation for NiTi, by Steve R Bailey in EuroPCR 2005). For example, the struts may be as small as 20 microns, or 10 microns in width and/or thickness.

Alternatively or additionally, the size and/or shape of the struts is selected to assist in embedding of the stent in the vascular wall. Optionally, the outside edges of the struts are machined to selectively embed (e.g., be sharp) or float (e.g., be flattened) on the intimal wall.

Optionally, strut number and/or total accumulated length is increased, without passing desired coverage of, for example, 10% or 15%. For example, the strut number may be at least 50, 100, 200, 250 or intermediate numbers for a 30 mm lengths stent. Similar numbers (scaled linearly) may be provided for other stent lengths, such as 10 mm, 15 mm, 20 mm, and 40 mm.

### Reduce Strut Number

Optionally or alternatively to making struts narrower, the number of struts is reduced (e.g., in mesh designs with many struts), as each strut can resist more radial compression of the stent. Optionally, the number of struts for a same stent resistance and general stent geometry and length, is reduced by at least 20%, 30%, 50%, 70% or intermediate amounts. Optionally, the cell size is greater than 0.1 mm², 0.2 mm², 0.25 mm², 0.3 mm². Optionally, the number of struts per mm length of the stent is less than 20, less than 10, less than 5.

### Elongation

A potential advantage of using thinner struts is that the elongation requirement of a narrow joint is generally smaller than that of a wide joint. In one example, described in greater length below, this allows a greater degree of opening of an intra-strut joint. In another example described below, this allows a same stent design to be implemented with a material with lower elongation, by trading off strength (and thus reducing width) with elongation properties. As noted below, a stent design may use partially cold-worked materials optimized for a certain elongation and strength needed by the stent.

In accordance with some embodiments of the invention, the relation between the maximum elongation and the bend radius may be modeled by:

εₘₐₓ=max(εₒᵤₜ,−εᵢₙ)

for:

$${ɛ}_{out} = \frac{h * ( \frac{{R}_{1}}{{R}_{1}} - 1 )}{2 * {R}_{1} + h}$$

strain on the bend exterior plane;

$${ɛ}_{i n} = - \frac{h * ( \frac{{R}_{1}}{{R}_{2}} - 1 )}{2 * {R}_{1} - h}$$

strain on the bend interior plane

### Where

h is the strut's width

R₁ is the hinge's neutral axis bending radius before bending

R₂ is the hinge's neutral axis bending radius after bending

In some embodiments of the invention, only the joints are made narrower and/or the joints are annealed and optionally made thicker, as required.

### Circumference Ratio and Intra-Strut Angle

One potential advantage of using thin struts is that a larger intra-strut angle may be achieved. Such larger angles can have one or both of the following potential advantages:

a) a greater diameter increase for a stent; and

b) a smaller spring-back of the stent as whole, possible even with materials that have a greater spring-back.

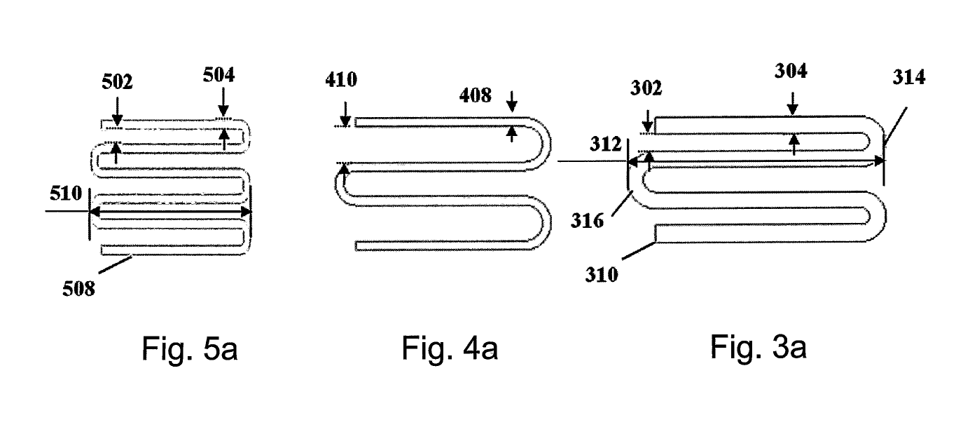

FIGS. 3A and 3B illustrate the opening of a strut in a stent having a low strut angle.

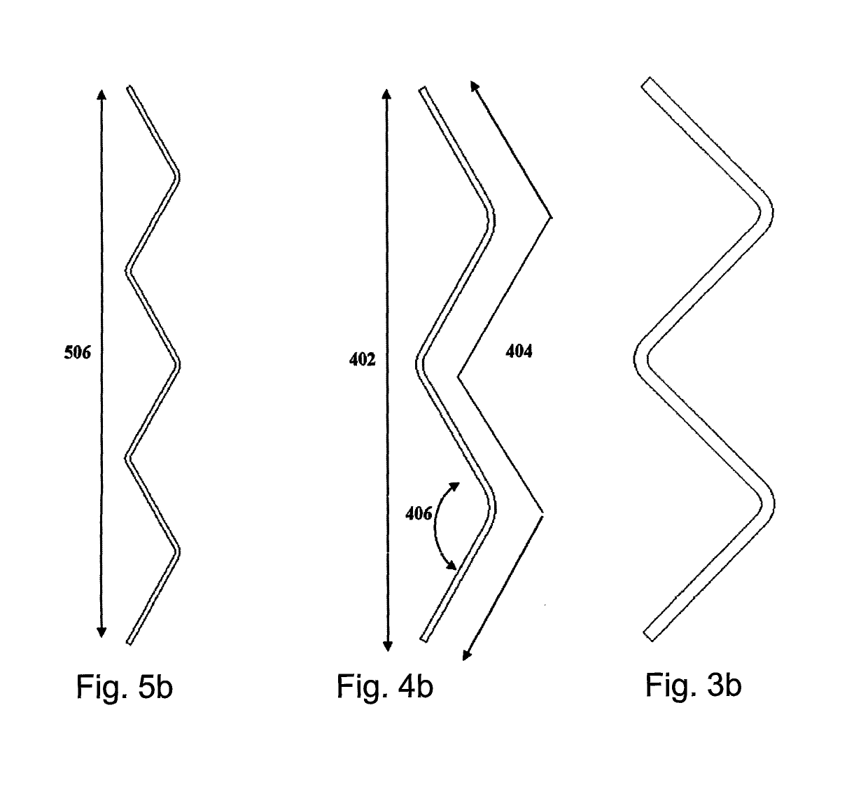

FIGS. 4A and 4B illustrate the opening of a strut in a stent having a low strut angle.

FIG. 3A shows a folded wide strut 310 (width shown is 304), which opens to a first strut shape as shown in FIG. 3B. FIG. 4A shows a narrow strut having a width 408 substantially smaller than width 304 (e.g., 0.05 mm as compared to 0.09 mm), which opens to a second strut shape in FIG. 4B, with an angle 406 between strut sections, which is larger than that achieved in FIG. 3B. For example, angle 406 can be 135 degrees as compared to 100 degrees. Optionally, the increase in angle from crimped to final shape is at least 120 degrees, at least 130 degrees, or at least 150 degrees.

A measure “circumference ratio” (CR) is an approximation of the cumulative effect of multiple intra-strut angles in a multi-segment strut, for example as shown in FIGS. 3 and 4. Circumference ratio is defined as the ratio between radial strut length 404 (i.e., along the strut segments/contour) and the distance between its two ends 402. In some stents, CR may be inversely proportional to the elastic to plastic curvature.

It should be noted, that under the mechanical principles of a deflecting beam, the narrower the strut is, the lower the designed CR could be. Meaning, in a stent with narrower struts higher inter-strut angles could be reached without forming any micro-cracks, or worse, completely fracturing the strut.

Low CR yields a high inter-strut angle 406 when the stent is expanded to its nominal diameter (FIG. 3b and FIG. 4b) from its crimped state (FIG. 3a and FIG. 4a), which can be advantageous when using a material which posses high spring back, such as materials having high yield strength or low Young's modulus.

A high inter-strut angle design is characterized, in some embodiments of the invention, by a circumference ratio of below 1.5, below 1.4, below 1.3, below 1.2, below 1.1. or intermediate values (all for the expanded stent).

It should be noted that the spring back is applied at the joints, and not to the strut as a whole. The larger the joint angle, the lower the effect on strut length, of a same size recoil, as shown in the table below, which illustrates in a relative manner the contribution to stent/strut recoil as a function of the expansion angle

| | Relative |
|---|---|
| angle | recoil |
| 80 | 0.766044 |
| 90 | 0.707107 |
| 100 | 0.642788 |
| 110 | 0.573576 |
| 120 | 0.5 |
| 130 | 0.422618 |
| 140 | 0.34202 |
| 150 | 0.258819 |
| 160 | 0.173648 |
| 170 | 0.087156 |
| 180 | 6.13E−17 |

This may be expressed mathematically as follows: recoil is inversely proportional required angular expansion of the stent by

$$Cos ( \frac{θ}{2} ) ,$$

where θ is the inter-strut angle. When the angle approaches 180 degrees, the effect of the cosine diminishes.

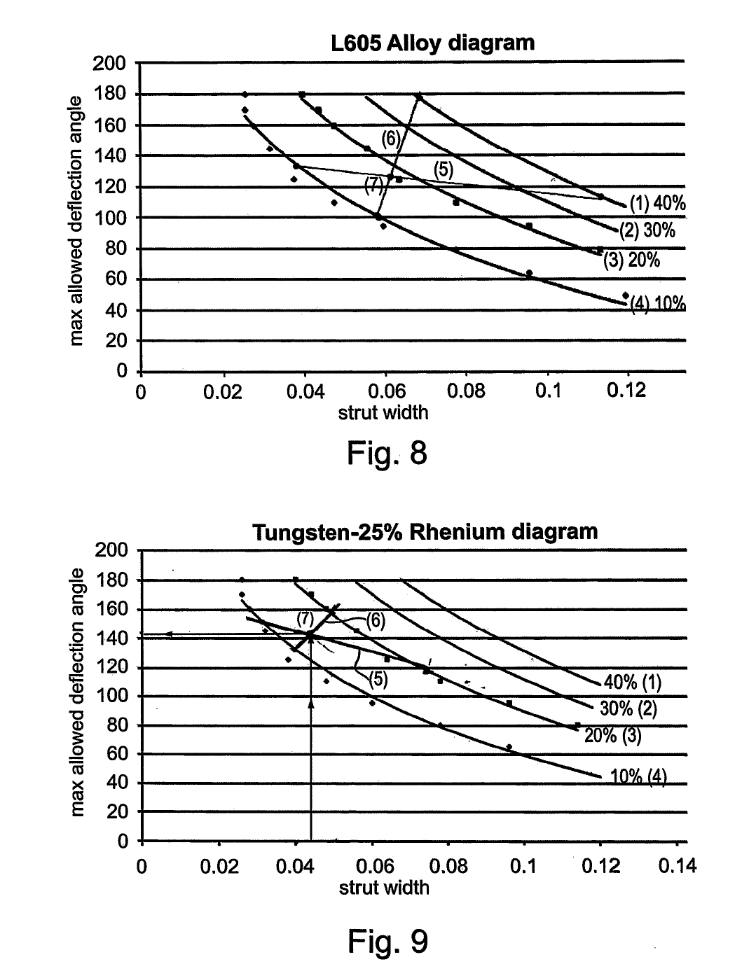

FIG. 7 is a graph illustrating how a low intra-strut angle can compensate for increased spring back, in accordance with exemplary embodiments of the invention. FIG. 7 shows, for two stents, a first is made from common stent material and the second is made from a high strength material (or low young's modulus) and designed to have a low CR.

In an exemplary embodiment of the invention, a lower CR is used to compensate for increased spring-back caused by using higher strength materials and/or materials with a lower young's modulus.

In an exemplary embodiment of the invention, the stent is characterized by the change in angle, not only by the final angle, for example, the change in angle may be, for example, 80, 100, 120, 140, 160, 180 or more degrees. An angle greater than 180 is possible when the initial angle is negative. A negative angle is optionally defined when the joint is wider than the distance between the far ends of the strut sections meeting at the joint, so that the joint forms the base of a triangle that is opened up (e.g., the two strut sections other than the base spread apart) by the expansion. Exemplary original (crimped) intra-strut angles can be, for example, −10, −20, −30, −40, −60, −90 degrees or greater or intermediate angles.

In an exemplary embodiment of the invention, the total number of joints for a 30 mm stents is less than 200, less than 100, less than 50. linear scaling may be applied to other stent sizes.

In an exemplary embodiment of the invention, at least 20%, at least 40%, at least 60%, at least 80% of all joints or of joints of radial struts, have the above described large joint angles.

### Changes in Fin Design

In an exemplary embodiment of the invention, usage of narrower struts allows different fin designs to be used. Fins are the portions of the struts that are folded in a generally axial direction and which change to a generally circumferential direction during expansion.

In accordance with one practical consideration, as the struts are narrower, there is room for more struts on a same circumference unit. In accordance with another practical consideration, many machining methods have limits on smallest clearance between two features possible. When struts are narrower, there is more room to machine between the struts.

In an exemplary embodiment of the invention, the stent design includes shorter fins and/or a greater number of fins, per circumferential unit.

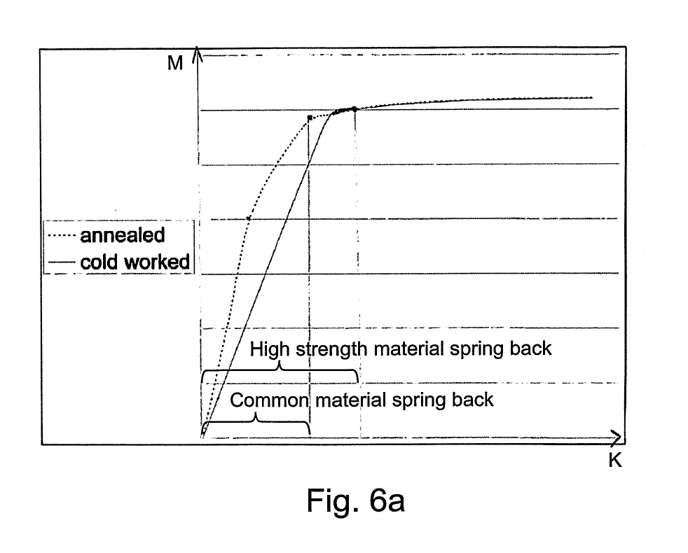

FIG. 5 shows a strut design with both more fins 508 and shorter fins. For any given radial strut, the fins may be of varying length and may interconnect with axial struts in various manners, for example, as known in the art.

### Fin Length

In an exemplary embodiment of the invention, fin length is reduced, optionally by providing a greater number of fins and/or by allowing each angle to open more and thus provide a greater diametric increase from a shorter overall linear length along the fins.

In an exemplary embodiment of the invention, fin length is considered as an average of the fins in the stent that take part (after expansion) in radial support. In an exemplary embodiment of the invention, fin length is 10%, 20%, 25%, 30%, 40% shorter that those of a stent of same general design and maximal expansion diameter. Optionally, fin length (510) is less than 1 mm, less than 0.8 mm, less than 0.7 mm, less than 0.5 mm or intermediate values.

In an exemplary embodiment of the invention, having a strut with short fins increased stent's deliverability, due to increased flexibility. Generally, an axially lying fin is difficult to bend during delivery, so longer fins define longer rigid units during delivery. As shown, a fin is a single segment 312 constrained between two hinge-like elements 314 and 316, within a radial strut.

In an exemplary embodiment of the invention, fin lengths are decided as (pi*Nominal Diameter*CR)/(Min. number. of fins), with a small safety factor (say 20%). For example, for a CR at 1.3, and 26 fins, the maximal fin lengths comes out:

| Nominal diameter | Fin maximal length |
|---|---|
| 2.5 mm | 0.47 |
| 3 mm | 0.56 |
| 3.5 mm | 0.65 |
| 4.0 mm | 0.75 |

### Fin Number Per Circumferential Unit

In an exemplary embodiment of the invention, a narrow strutted stent is designed with a greater number of fins than existing designs, for example, 15%, 20%, 30%, 40%, 50%, 60%, 80%, 100% or greater or intermediate additional fins. For example, for a coronary stent, more than 25, more than 26, more than 30, more than 35 or intermediate numbers of fins may be provided. Optionally, fewer than 100, fewer than 60, fewer than 50 fins are provided in a circumferential unit.

In an exemplary embodiment of the invention, increasing the number of fins allows designing a stent with a greater expansion ratio between crimped and expanded state. Such a stent may be useful for large peripheral vessels, such as veins and arteries.

It should be noted that, typically, the number of fins is limited by the manufacturing process of stents, cut from tubing, due to a requirement for minimal clearance 410 between the fins when the profile is being cut (e.g., for laser and e-beam cutting). When using a narrower strut design, the clearance between the fins increases 410 compared to previous strut design 302. Therefore, reducing strut's width 504, while keeping the clearance between the fins at a minimum process parameter 502, will allow an increase in the total number of fins per radial strut.

In an exemplary embodiment of the invention, a radial strut is provided with two or more levels of joints, for example, the strut may be defined by a plurality of strut segments connected by joints, each such segment being expandable as well, for example, being formed of a plurality of sub-segments connected by joints. In another design, the strut segments are wavy sections of metal that can be straightened by application of force (e.g., with an inflation balloon).

In an exemplary embodiment of the invention, the number of joints is approximately the number of fins, for example, within a factor of 2, 3 or 4. Optionally, the joints are annealed more than other parts of strut, but the opening of the joint cold-works it and strengthens it. Optionally, joints are defined by making them more prone to deformation, for example, due to shape, width and/or weakenings associated therewith, for example, using methods known in the art.

### Cold Worked Materials

In general, the methods and designs described above are suitable for every material having high yield strength and in particular for materials having yield strength of more than 1000 MPa, more than 1500 MPa, or more than 2000 MPa. Generally, however, such materials need to have minimal ductility to allow bending of the struts upon stent expansion. In contrast to common practice of using annealed materials, in some embodiments of the present invention, the materials used are partly or completely cold-worked, even though it reduces their elongation properties. Optionally, methods as described above (e.g., narrow strut) compensate for reduced elongation.

For many materials, the material can be along a scale that spans form completely annealed (maximum elongation) and completely cold worked (minimum elongation. In an exemplary embodiment of the invention, the material is cold worked to be along this scale, optionally, with sufficient elongation for the stent designed needs.

In an exemplary embodiment of the invention, the material is cold worked so that it still maintains an elongation of at least 10%, 15%, 20%, 25%, 30% or intermediate numbers. The exact number will typically depend on the needs as defined by the final diameter, strut width and stent design (e.g., intra-strut angle). The range of elongation between the minimum and maximum is termed herein elongation range. Optionally, at about 20%, about 40%, about 60%, about 70%, or intermediate percentages of this range are maintained by the cold working.

It should be noted that as the stent is made of thin-walled tubes, very high cold working can generally be achieved. Optionally, the actual degree of cold working is measured during manufacture, for example, statistically, as part of a quality assurance process.

In some embodiments of the invention, “standard” stent materials are used, albeit in a cold worked state, for example, stainless steel and cobalt-chrome alloys having a yield strength of about 800 MPa or more (e.g., 1000 MPa or more), with an elongation of about 20% or more (e.g., before cold-working).

### Exemplary Stent Materials

In some embodiments of the invention, various alloys are used, for example, Rhenium alloys, optionally cold worked. It should be noted that the materials described herein are cold-worked in only some embodiments of the invention and are annealed in others.

In an exemplary embodiment of the invention, Rhenium alloys, with the following characteristics (compositions are in atomic percentage), are used:

a) Tungsten Rhenium—In order to achieve desired room temperature ductility values rhenium content within the alloy is optionally kept above 20%, and optionally between 23% and 28%. As a general guideline, Rhenium content should be kept to maximum, under manufacturer's ability to maintain full solubility of Rhenium within the Tungsten at solid state (i.e. σ-phase should be avoided in some embodiments of the invention, even at the expense of reduced Rhenium content). Usually in commercial industry this value will not exceed 26% Rhenium. Although stents could be formed from Diluted Rhenium as well, the Rhenium rich alloys in some embodiments of the invention exhibit significantly desirable stent properties such as high room temperature ductility, higher yield strength and high fracture resistance.

b) Rhenium-Tungsten—Alloys with Tungsten content of 0.1% to 10% and 99.9% to 90% rhenium. The alloy can have a relatively high ductility when annealed (35% to 44%), which can ease manufacturing processes. When annealed the material has a yield strength of about 600 to 650 MPa, and is assumed to have a yield strength of more than 1000 MPa when cold worked to an elongation of 15% to 20%

c) Molybdenum-Rhenium—Though this alloy may possess slightly lower strength than a Rhenium rich Tungsten alloy, this alloy is typically more ductile. In order to maximize the rhenium alloying effects of improved ductility and strength Rhenium content optionally exceeds 10%, 20%, 30% or more or intermediate percentages, up to Rhenium's maximum solubility limit, which is around 50%. Optionally, some of the increased ductility is traded off for strength by cold-working, as described herein. It is noted that ductility can assist during manufacture.

In some embodiments alloys similar to to above are used, with the addition of metal-carbides or metal-oxides. These additives may suppresses the growth of recrystalized grains during annealing, which allows maximum ductility of the material. Such substances could be Zirconium-Carbide, Hafnium-Carbide, or Thorium-Oxide with up to 2% of the weight composition.

In some embodiments small amounts of Osmium are added to further increase the positive influence of Rhenium. Optionally, the Osmium and/or other additives are added at the expense of the non-Rhenium component, at the expense of Rhenium or as an addition to the total mixture.

In some embodiments of the invention, it is desired to use materials having approximately 2000 MPa, or more. In some embodiments, cold-working and/or low temperature annealing of the materials described is used in order to achieve desired strength.

Another material property desirable in some embodiments of the invention, is small grain size, desirably under 10 microns, or under 5 microns, which size may improve strength, for example, by 10%, 15%, 20% or more, compared to high strength material.

The following table shows various stent materials and their properties. The Minimal elongation to break is for cold worked materials, with the last number (where shown) is a typical number. The Maximum strut width is the maximum recommended strut width for the design shown, when the final diameter is as in the table, for a cold worked material.

| | | | | Min. | | | | | |
|---|---|---|---|---|---|---|---|---|---|
| | | Annealed | Cold | elongation | Max. Strut | | | | |
| | Annealed | elongation | worked | to | width up | | | | |
| | strength | to break | strength | break | to 2.5 mm | 3 mm | 3.5 mm | 4.5 mm | 6 mm |
| 316 | About | 50% to 60% | About | 10% | 0.06 | 0.065 | 0.07 | 0.075 | 0.08 |
| stainless | 400 MPa | | 750 MPa | to | | | | | |
| steel | | | | 20%. | | | | | |
| | | | | 15% | | | | | |
| Co—Cr | About | 35%-55% | About | 10% | 0.06 | 0.065 | 0.07 | 0.075 | 0.08 |
| alloys | 500 MPa | | 800 MPa | to | | | | | |
| | | | | 20%. | | | | | |
| | | | | 15% | | | | | |
| Biodur | About | ~50% | About | 10% | 0.05 | 0.055 | 0.06 | 0.065 | 0.07 |
| 108 | 610 MPa | | 1250 Mpa | to | | | | | |
| | | | | 20%. | | | | | |
| | | | | 15% | | | | | |
| WRe | 1300 MPa | Up to 20% | 2000 to | 10% | 0.045 | 0.05 | 0.055 | 0.06 | 0.065 |
| | | | 2500 MPa | to | | | | | |
| | | | | 15%. | | | | | |
| | | | | 12% | | | | | |
| ReW | About | 37% | About | Less | 0.045 | 0.05 | 0.06 | 0.065 | 0.07 |
| | 620 MPa | | 1100 to | than | | | | | |
| | | | 1400 Mpa | 10% | | | | | |
| MRe | 850 MPa | Up to 25% | About | 10% | 0.045 | 0.05 | 0.055 | 0.06 | 0.065 |
| | | | 1500 to | to | | | | | |
| | | | 1800 MPa | 15%. | | | | | |
| | | | | 12% | | | | | |

It should be noted there are additional materials in each class (e.g., there are more types of stainless steel than just 316LS and Biodur108 and more types of Co—Cr alloys besides L605 and more types of Tungsten-Rhenium alloys).

It should be noted that cold working to below 10%, is possible, but not desirable for embodiments where a minimum elongation is needs to support strut joint deformation. In general, as elongation goes down to those values, additional benefits of further strut thinning decrease while cost due to intra-strut angle limits continue raising.

Following is a (non-exhaustive) list of materials which may be used for stent production (cold worked or not) in accordance with exemplary embodiment of the invention:
(a) stainless steels including:

316L, 316LS, 316LVM

Fe-21Cr-10Ni-3.5Mn-2.5Mo ASTM F 1586

Fe-22Cr-13Ni-5Mn ASTM F 1314

Fe-23Mn-21Cr-1Mo-1N Nickel free SS (BIODUR108 alloy Carpenter (USA))
(b) Co—Cr alloys including:

Co-20Cr-15W-10Ni: “L605” ASTM F90

Co-20Cr-35Ni-10Mo: “MP35N” ASTM F 562

Co-20Cr-16Ni-16Fe-7Mo Phynox ASTM 1058

Co—Cr with added Rhenium, of up to 5%, 10%, 15% or 20%
(c) Refractory alloys (all ratios are in weight percentages NOT atomic)
   a. Binary composition (alpha phase)
      i. tungsten 70% to 99%, optionally 70% to 80%
      ii. Rhenium 1% to 30%, optionally 20% to 30%
   b. Binary composition (beta phase)
      i. Tungsten 0% to 15%, optionally 0.01% to 10%.
      ii. Rhenium 100% to 85%, optionally 99.9% to 90%
      iii. Pure Rhenium may be used as well
   c. A composition with
      i. Up to 30% rhenium
      ii. Up to 5% Osmium, optionally 1% to 2% Osmium
      iii. Balance tungsten (65% to 100%)
   d. A composition as described herein with at least one of the following:
      i. Hafnium or Hafnium-carbide (up to 1%, 2%)
      ii. Zirconium or Zirconium-Carbide (up to 1%, 2%)
      iii. Niobium (up to 5%, 10%)
      iv. Yitrium (up to 5%, 10%)
      v. ThO2 (Thorium Oxide) up to 5%, 10%
   e. A ternary composition of Tungsten-Rhenium-Molybdenum
      i. 25% to 45% rhenium
      ii. Any combination for the balance of Tungsten and Molybdenum, for example, up to 20%, 30% of either
      iii. Additives as in d.
   f. A Composition with
      i. Rhenium of 0% to 50%, and preferably 30% to 50%, or 5% to 10%.
      ii. Balance by Molybdenum

In addition, also considered are any alloy with up to 5%, 10%, 15%, 20% or intermediate or greater percentages of Rhenium, for example, stainless steel or Co—Cr with added Rhenium.

The following table shows the exemplary relationship between elongation and yield strength for various materials it should be noted that variations of up to 20%, or more may occur depending on the manufacturer and dimensions of the material.

| | | | | | ReW5 | Mo-47.5Re |
|---|---|---|---|---|---|---|
| | | L605 (a | | | (rhenium | (Molybdenum |
| | | Co—Cr | Biodur108 | | tungsten | with 47.5% |
| | 316ls | alloy) | (carpenter) | WRe25/26 | 5%) | rhenium) |
| Young's | 200 GPa | 240 GPa | 200 GPa | 400 GPa | 450 GPa | 365 GPa |
| Modulus | | | | | | |
| Elongation | Yield strength [MPa] | | | | | |
| 10% | 900 | 900 | 1598 | 2400 | ~1200 | ~1700 |
| 20% | 780 | 820 | 1190 | 1300 | ~1000 | 845 |
| 30% | 650 | 780 | 952 | — | ~800 | |
| 40% | 530 | 600 | 680 | — | 625 (at | |
| | | | | | 37% | |
| | | | | | elongation) | |
| 50% | 400 | 500 | 612 | — | — | |

Young Modulus

FIG. 6B is a stress-strain diagram of some materials which may be used for manufacturing stents, in accordance with exemplary embodiments of the invention. It should be noted that some of the materials described herein have a high Young's modulus, so that a lower spring-back is expected. This may allow lower intra-strut angles to be used, if desired. For example, Rhenium-tungsten has a strength similar to stainless steel, but double the young modulus. Conversely, materials with low Young's modulus, are optionally compensated for by using higher-intra-strut angles, so that the effect of spring-back is reduced. Optionally, young modulus values above 300 GPa, above 400 GPa above 600 GOa or more are useful. Conversely, the stent design can tolerate low Young modulus values, for example, smaller than 150 GPa, smaller than 100 GPa, smaller than 50 GPa, for example, by suitable selection of intra-strut angles.

### Exemplary Fabrication Method

Stents can be fabricated, for example, from corrugated wires of the chosen material or from a slotted tube (e.g., with a diameter of less than 2 mm for stent of a diameter of up to 5 mm). Methods for producing stainless steel stents and Cobalt-Chromium stent are well known in the art.

In an exemplary embodiment of the invention, stents are produced from Rhenium alloys (or other high yield strength materials) by:

(a) Semi-hardened Tubes are produced from a sintered tubeshell, which is then extruded, drawn and swaged to its final dimension.

(b) Thin strips are rolled from sintered ingots, which are then bent and welded by electron-beam, stress relieved by shot-pinning and reduced by drawing.

In an exemplary embodiment of the invention, an alternative method is provided. This method may assist in overcoming limitations caused by high work hardening coefficient limits of the material and/or the high complexity of producing small diameter tubing by bending foils of this kind of materials. In an exemplary embodiment of the present invention a cold worked Rhenium alloy tube is produced by sintering or melting either a full rod or a semi-finished tubing. Following, the semi finished product is optionally cold-worked and/or annealed to achieve desired mechanical properties as described herein. Following, the inner outer surfaces of the tubing are optionally machined to reach the final dimensions. Following, production is performed, for example by laser cutting and optional electro-polishing of the finished stent.

Alternatively or additionally, a stent may be formed using vapor deposition, sputtering, or other material adding methods known in the art. Alternatively or additionally, the stent is formed by etching (e.g., chemical/light, plasma or e-beam) of a form, such as on a tube.

### Stent Variations

The above description has focused on radial load bearing struts in stents. Optionally, a stent is manufactured in which at least 60%, at least 70% , at least 80% of the material bearing radial forces (or of the materials bearing at least 60%, 70%, 80% of the radial forces) complies with the above methods and designs. Optionally, the stent includes a covering of, for example, metallic mesh. Optionally, this mesh does not necessarily follow the above methods and designs. Optionally, any such mesh has a structure which is not aligned with the force bearing struts.

In actual design, due to, for example, inaccuracies or manufacturing constraints, optimal values as described herein may not be reached. Optionally, a stent is designed to within 10% or 20% of an optimum value.

While a stent may be formed of a single material, in some embodiments, the stent is formed of multiple materials, for example, radial struts may be made of a different material than axial struts. In another example, the stent is formed of layers. Optionally or alternatively, the stent is formed of a core with a coating. In an exemplary embodiment of the invention, the stent is formed of 2, 3 or more materials, some of which may be provided for strength and other for other reasons, such as biocompatibility or dug elution. Optionally, the stent is coated, for example, with a radioactive coating, with a drug eluting coating, with a thrombogenic (or anti-) coating, with an anti-inflammatory coating and/or with a coating to enhance neo-intimal growth. Other coatings as known in the art of stents may be used as well.

The stent and/or struts may have a fixed thickness or the thickness may vary, for example, be greater at joints, or greater for radial struts. Optionally, thickness is lower than width at least at some points, for example, along struts where there is no joint and/or otherwise less risk of bending out of the stent surface.

The width of struts may vary, for example, be increased or decreased at joints or between joints.

Optionally, different parts of the stent have different degrees of cold-working, for example, at least 2, at least 3, at least 4 or more different degrees of cold working. Optionally, the stent is selectively annealed, for example, using an e-beam or using local heating, for example, at points where a high elongation is needed (e.g., at joints or at outer or inner bends thereof). Optionally or alternatively, axial struts are annealed more, especially at places where they join radial struts.

In an exemplary embodiment of the invention, the number of axial struts is reduced (e.g., by 20%, 30%, 50% as compared to a similar stainless steel annealed stent of same original geometry), due to increased strength provided by the material. Alternatively or additionally, the width of the axial struts is reduced. Optionally, an open cell design is used.

### Example for Applying Design Principles on Existing Stents

In an exemplary embodiment of the invention, existing stent designs are modified using to use materials and/or geometries as described herein. By existing designs it is meant the general layout of struts and manner in which struts interact to provide radial and/or axial support in a completed stent and deliverability (e.g., crimpability and flexibility) in a collapsed form.

In an exemplary embodiment of the invention, an existing stent design is modified by changing material properties and thus strut width. Intra-strut angle, fin length fin spacing, and/or fin number, are optionally modified to make use of change in stent properties and/or to provide greater diameter.

In other methods, radial struts may be deleted, for example, by at least 10%, 20%, 30%, 40% or intermediate numbers, for example, by spacing radial struts further apart axially. Optionally, cell sizes are maintained by adding axial struts.

In an exemplary embodiment of the invention, the stent is designed to have, one, two, three, or more of the following characteristics:

(a) Radial force high enough to keep the blood vessel lumen open.

(b) Minimal wall thickness to achieve less potential restenosis with improved insertion profile.

(c) Thin struts to allow a minimal metal-tissue coverage area percentage, while optionally maintaining standard cell cross-section area.

(d) Using an alloy with elongation properties matching the stent design, to allow stent expansion without developing micro-cracks.

(e) Using a biocompatible material, having minimal (or no) Nickel and/or Molybdenum contents.

(f) Allowing metal deformation of the stent without passing alloy's maximal elongation capability, while allowing only minimal spring back of stent (e.g., less than 10%, less than 5%, less than 3%, less than 2%, less than 1%) after expansion.

An illustrative example of applying the design guidelines of the present invention is given based on the Multi-Link Vision stent (Manufactured by Guidant Corporation, Menlo Park, Calif.). The stent features a cobalt chrome strut with a thickness of 0.08 mm and an inter-strut angle at nominal diameter of about 90 degrees, which is correlated to a CR of 1.42. By applying the guidelines the strut thickness and width is reduced by 50% to 0.04 mm. Inter-strut angle is increased to 140 degrees, and radial strut fin length is reduced by 25%, while maintaining CR. The resulting modified stent features the same radial strength and blood vessel scaffolding. However, through these changes, compared to the original stent design, metal coverage area of the modified stent is reduced by 65% to about 4.5% (compared to about 13%), stent delivery profile is reduced by at least 10% to less than 0.9 mm (compared to 1 mm), with the possibility of an additional reduction due to increased clearance between adjacent strut fins.

### Exemplary Method of Stent Design

Following is described a graphical method (which may be applied mathematically), for finding, for a given material, an optimal strut width and cold working degree. Optionally, the method is applied using finite element analysis or other computerized mechanical analysis methods.

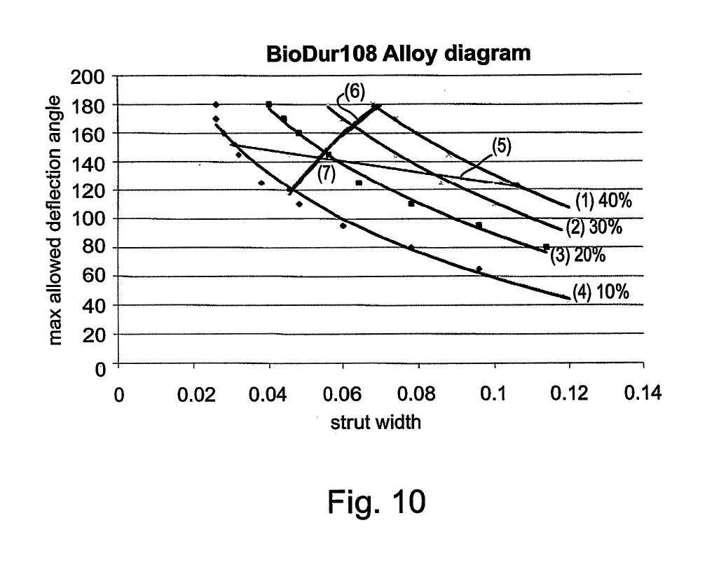

FIGS. 8-10 are exemplary charts drawn using the methods described below, for SS, Cr—Co and WRe. The base lines of the chart are based on a geometrical relation (therefore generally material independent). Each line reflects a relation between the strut's width and the maximal allowable deflection angle given material's elongation to break. In the charts lines were drawn for: (1) 40% elongation ; (2) 30% elongation ; (3) 20% elongation and (4) 10% elongation. More lines could be drawn within intermediate ranges, or on a wider scale.

The lines where drawn based on the following equation

$$ɛ = - \frac{h * ( \frac{{R}_{1}}{{R}_{2}} - 1 )}{2 * {R}_{1} - h}$$

### Where:

h is the strut's width

E is the maximum elongation

R₁ and R₂ are the radiuses of curvature before and after bend was applied, respectively.

Thus, the angle of deflection can be calculated.

Following 2 more lines are drawn on the sketch:

1. A “fixed recoil” line.
a. For a material in annealed state—design a stent having a specific recoil and nominal expansion angle θ₁. Optionally, an existing design having various useful properties is used. In an alternative embodiment, the material and its properties are a given and the stent is directly designed to be usable when constructed using these materials.
b. Based on material strength properties dependency on, for example, cold-working and/or Young's Modulus, set a relation between elongation and minimal deflection angle θ₂ to maintain desired Recoil set on (a), using the following relation

$$\frac{{YS}_{1}}{{E}_{1}} * cos ( {θ}_{1} ) = \frac{{YS}_{2}}{{E}_{2}} * cos ( {θ}_{2} )$$

Where:
   YS₁ , E₁ are the stent's material properties used for initial design (a)
   YS₂ , B₂ are the stent's material properties used for high strength design achieved by high-strength material selection and/or cold-working and could be reflected by a specific elongation value per specific material
c. Draw a line (5) based on the above relation (b).

2. An “elongation strut-width” line:
a. Derive strut's width H₁ for the stent designed in 1-a made from a material in the annealed state
b. Based on material strength and/or elongation properties recalculate minimal required width H₂ in order to maintain radial force of the stent designed in 1-a, using the following relation:

YS₁*H₁³=YS₂*H₂³
c. Draw a line (6) based on elongation (derived from YS) and strut width
d. It should be noted that the target radial force may be changed as well.

3. Analysis of the diagram:
The intersection (7) of lines (5) and (6) is the minimal elongation/maximal cold reduction point for the material used for the stent. Since it reflects an optimum between width reduction while maintaining desired recoil properties.
Width of stent refers to sections where the deflection occurs upon stent expansion.
If higher recoil is acceptable, the deflection angle could be reduced, yielding an allowed higher degree of cold working and, possibly lower strut width. In some cases, lower recoil is desired, so intra-strut angle may be increased, optionally requiring more elongation and less cold-working (or a different material).

FIGS. 8-10 show 3 diagrams constructed based on the above method. The results for the intersection point are shown in the following table:

| | | | Nominal | |
|---|---|---|---|---|
| | | Yield | deflection | |
| Material | Elongation | strength | angle | CR |
| W-25% Re | 13% | 1800 MPa | 140 degrees | 1.1 |
| L605 (Co—Cr alloy) | 18% | 830 MPa | 125 degrees | 1.15 |
| BioDur108 (Carpenter, | 18% | 1200 MPa | 140 degrees | 1.1 |
| USA) | | | | |

It should be noted that the above method was illustrated and explained on stent with a constant strut width, and straight fin geometry. However, the same design approach can be applied on different stent geometries, including but not limited to, varying width, curved fins, open cell stent, and stents having a circular or ellipsoid cross-section supporting elements (rather than rectangular as shown).

Typically, as part of good engineering practice safety margins are applied to the results, which may yield less than optimal designs. Exemplary requirements are fatigue resistance, allowance for over expansion and/or manufacturing dependent tolerances.

Optionally, after design is completed it is tested. Optionally, an iterative design approach is used, in which the design is modified (optionally using the methods described herein, after testing. Optionally, the design is modified to fine tune deliverability, radio-opacity and/or matching to delivery system of the stent.

### Non-Coronary Support Structures

The above description has focused on coronary stents which are characterized by particular deliverability, strength and/or expansion requirements. The methods described herein may, however, be applied to other stents, such as cerebral stents, urethral stents, peripheral vascular stents, venous stents, biliary stents, vascular stent-grafts and/or stents and ductual supporting elements for other parts of the body. Exemplary final diameters of stents are less than 30 mm, less than 20 mm, less than 10 mm, less than 5 mm, less than 2 mm and less than 1 mm. it is noted in particular that reducing stent material and fin length may be especially useful in treating torturous blood vessels, such a sin the brain or narrowed coronary vessels. However, for the ease of explanation, a detailed description of a coronary stent is utilized.

### General

Various designs for stents and stent delivery systems are known in the art. It should be appreciated that various ones of the statements described herein may be adapted for such stents and/or delivery devices, in accordance with exemplary embodiments of the invention.

It will be appreciated that the above described stents and methods of stent design may be varied in many ways, including, omitting or adding steps, changing the order of steps and the types of devices used. In addition, a multiplicity of various features, both of method and of devices have been described. In some embodiments mainly methods are described, however, also apparatus adapted for performing the methods are considered to be within the scope of the invention. It should be appreciated that different features may be combined in different ways. In particular, not all the features shown above in a particular embodiment are necessary in every similar embodiment of the invention. Further, combinations of the above features are also considered to be within the scope of some embodiments of the invention. Also within the scope of the invention are kits which include sets stents mounted on delivery systems. Also, within the scope is hardware, software and computer readable-media including such software which is used for carrying out and/or guiding the steps described herein, such as stent design. Section headings are provided for assistance in navigation and should not be considered as necessarily limiting the contents of the section. When used in the following claims, the terms “comprises”, “includes”, “have” and their conjugates mean “including but not limited to”. It should also be noted that the device is suitable for both male and female, with male pronouns sometimes being used for convenience.

It will be appreciated by a person skilled in the art that the present invention is not limited by what has thus far been described. Rather, the scope of the present invention is limited only by the following claims.

1. A stent formed of at least 60% of a material that is cold-worked to an extent of at least 5%.

2. A stent utilizing a plurality of radial struts to provide structural support to a blood vessel, wherein at least 60% of the radial struts from which the stent is constructed are formed of a material that is cold-worked to an extent of at least 5%.

3. A stent according to claim 1, wherein said extent is at least 20%.

4. A stent according to claim 1, wherein said extent is at least 30%.

5. A stent according to claim 1, wherein said extent is at least 40%.

6. A stent according to claim 1, wherein said stent has at least one radial strut, wherein when said stent is expanded to a maximal predetermined diameter, said radial strut has an elongation of less than 30% applied thereto.

7. A stent according to claim 6, wherein said elongation is less than 20%.

8. A stent according to claim 6, wherein said elongation is less than 15%.

9. A stent according to claim 6, wherein said elongation is at least 10%

10. A stent according to claim 1, wherein said material is stainless steel.

11. A stent according to claim 1, wherein said material is an alloy comprising Tungsten and Rhenium at percentages above 5% each.

12. A stent according to claim 1, wherein said material is an alloy comprising Molybdenum and Rhenium at percentages above 5% each.

13. A stent according to claim 1, wherein said stent as a whole springs back radially less than 10% when expanded to a maximum design diameter thereof.

14. A stein according to claim 1, wherein said stent as a whole springs back radially less than 5% when expanded to a maximum design diameter thereof.

15. A stent according to claim 1, wherein said stent is a coronary stent.

16. A stent according to claim 1, wherein said stent is a cerebral stent.

17. A stent according to claim 1, wherein said stent has a maximum design diameter of less than 3.5 mm.

18. A stent according to claim 1, wherein said stent has a maximum design diameter of less than 2.5 mm.

19. A stent according to claim 1, wherein said stent has a maximum design diameter of less than 4.5 mm.

20. A stent according to claim 1, wherein said material is stainless steel, said stent is designed for a nominal expansion diameter of 2.5 mm or less and wherein said stent is formed mostly of weight bearing struts having a width of about or less than 0.05 mm.

21. A stent according to claim 1, wherein said material is stainless steel, said stent is designed for a nominal expansion diameter of 3.5 mm or less and wherein said stent is formed mostly of weight bearing struts having a width of about or less than 0.06 mm.

22. A stent according to claim 1, wherein said material is a tungsten-rhenium alloy, said stent is designed for a nominal expansion diameter of 2.5 mm or less and wherein said stent is formed mostly of weight bearing struts having a width of about or less than 0.035 mm.

23. A stent according to claim 1, wherein said material is a tungsten-rhenium alloy, said stent is designed for a nominal expansion diameter of 3.5 mm or less and wherein said stent is formed mostly of weight bearing struts having a width of about or less than 0.045 mm.

24. A stent according to claim 1, wherein said stent comprises a plurality of struts, and wherein at least 40% by length of said struts have a width of less than 0.05 mm.

25. A stent according to claim 24, wherein said 40% of struts have a width of less than 0.04 mm

26. A stent according to claim 24, wherein said 40% of struts have a width of less than 0.03 mm.

27. A stent according to claim 1, wherein said stent has a maximum design diameter which is at least 200% a crimped diameter.

28. A stent according to claim 1, wherein said stent has a maximum design diameter which is at least 300% a crimped diameter.

29. A stent according to claim 1, wherein said stent has a maximum design diameter which is at least 400% a crimped diameter.

30. A stent according to claim 1, wherein said stent, at a maximum design diameter thereof, has a wall coverage of less than 10%.

31. A stent according to claim 1, wherein said stent, at a maximum design diameter thereof, has a wall coverage of less than 6%.

32. A stent defining a cylindrical surface and having a nominal maximum expansion diameter and having a ratio between said surface and a surface area of said stent of more than 1:10, at said diameter.

33. A stent according to claim 32, wherein said ratio is greater than 1:15.

34. A stent according to claim 32, wherein said ratio is greater than 1:20.

35. A stent according to claim 32, wherein said diameter is less than 4 mm.

36. A stent according to claim 32, wherein said diameter is less than 3 mm.

37. A stent according to claim 32, wherein said stent is a coronary stent.

38. A stent according to claim 32, wherein said stent is a cerebral stent.

39. A stent according to claim 32, wherein said stent is a peripheral vessel stent.

40. A stent according to claim 32, wherein said stent is a non-vascular stent.

41. A stent formed of a plurality of expandable radial struts which provide at least 60% of radial support to said stent, wherein a width of at least 40% of said struts is less than 60 microns.

42. A stent according to claim 41, wherein said struts have a thickness about equal to said width.

43. A stent according to claim 41, comprising an additional mesh of thin-diameter material.

44. A stent according to claim 41, wherein said struts have a width of at least 10 microns.

45. A stent according to claim 41, wherein said struts have a width of at least 15 microns.

46. A stent according to claim 41, wherein said struts have a width of less than 50 microns.

47. A stent according to claim 41, wherein said struts have a width of less than 37 microns.

48. A stent according to claim 41, wherein said struts have a width of less than 30 microns.

49. A stent according to claim 41, wherein said struts have a width of less than 20 microns.

50. A stent according to claim 41, wherein said stent provides vascular support at least matching a gold standard of support.

51. A stent according to claim 50, wherein said struts are less than 60% the cross-section of struts of a same material in annealed condition, needed for providing said standard for said stent design.

52. A stent according to claim 50, wherein said struts are less than 60% the cross-section of struts of stainless steel 316LS, needed for providing said standard for said stent design.

53. A stent according to claim 52, wherein said cross-section is less than 40% of said cross section needed for SS.

54. A stent according to claim 52, wherein said cross-section is less than 30% of said cross section needed for SS.

55. A stent according to claim 52, wherein said cross-section is less than 20% of said cross section needed for SS.

56. A stent formed of a plurality of folded radial struts, said struts in folded condition defining fins, at least 80% of said fins having an axial length along the stent axis of less than ¼ of a nominal diameter of said stent.

57. A stent according to claim 56, wherein said length is less than ⅕ said diameter.

58. A stent according to claim 56, wherein said length is less than ⅙ said diameter.

59. A stent according to claim 56, wherein said length is less than 1/7 said diameter.

60. A stent according to claim 56, wherein said length is correct for at least 90% of said fins.

61. A stent having a designed maximum diameter of less than 3 mm, formed of a plurality of folded radial struts, each folded section being a fin, and having more than 26 fins in a circumferential direction for at least one axial section of the stent.

62. A stent according to claim 61, and having more than 30 fins.

63. A stent according to claim 61, and having more than 35 fins.

64. A stent according to claim 61, and having more than 40 fins.

65. A stent according to claim 61, and having more than 45 fins.

66. A stent according to claim 61, and having at least three such axial sections which do not overlap.

67. A stent formed of a plurality of folded radial struts, including expansion joints, at least 30% of said joints being designed to straighten to an intra-strut angle of greater than 120 degrees.

68. A stent according to claim 67, wherein said angle is greater than 130 degrees.

69. A stent according to claim 67, wherein said angle is greater than 150 degrees.

70. A stent according to claim 67, wherein said percentage is greater than 50%.

71. A stent according to claim 67, wherein said percentage is greater than 70%.

72. A stent formed of a plurality of folded radial struts, at least 40% of said struts being designed to straighten so that a ratio between a physical length of the strut and a linear length between ends of the strut is less than 1.4.

73. A stent according to claim 72, wherein said ratio is less than 1.26.

74. A stent according to claim 72, wherein said struts effect an elongation of about or less than 20% when straightening out to a maximum design diameter.

75. A stent according to claim 72, wherein said at least 40% of struts comprise at least 60%.

76. A stent according to claim 72, wherein said at least 40% of struts comprise at least 80%.

77. A stent according to claim 72, wherein said stent is an arterial stent with a final diameter 3.5 mm or less.

78. A stent formed of an alloy including Tungsten and Rhenium, wherein a percentage of Rhenium is greater than about 25%.

79. A stent according to claim 78, wherein said percentage is greater than about 26%.

80. A stent according to claim 78, wherein said Rhenium is in a percentage of greater than 50%.

81. A stent according to claim 78, wherein said Rhenium is in a percentage of greater than 80%.

82. A stent according to claim 78, wherein said alloy includes up to 5% of one or more additives selected form the group of Osmium, Carbides and Oxides.

83. A stent comprising a structure, said structure being fowled of at least 80% by volume of a radio-opaque material more opaque than stainless steel 316, said structure being less radio-opaque than by a factor of at least 1.4 than a stent of same geometric design formed of stainless steel 316 with strut cross-sections suitable for a stainless steel design.

84. A stent according to claim 83, wherein said stent includes at least one radio-opaque marker.

85. A stent according to claim 83, wherein said factor is at least 2.

86. A method of manufacturing a stent, comprising:
forming a tube of a metal or an alloy thereof;
cold working the tube; and
cutting said tube to form said stent.

87. A method according to claim 86, comprising machining said tube to size.

88. A method of stent design, comprising:
(a) providing a stent design including a plurality of struts;
(b) modifying a material property of said design;
(c) reducing a strut width for at least 40% of said struts; and
(d) modifying at least one of intra-strut angle, strut CR, number of fins and length of fins in a manner which maintains a diametric recoil in a same nominal diameter at less than 5%, and maintains a same minimal required radial resistance

89. A method according to claim 88, wherein said (b) comprises selecting a degree of annealing.

90. A method according to claim 88, wherein said (b) comprises selecting a degree area reduction each of the tubing production phases.

91. A method according to claim 88, wherein said (b) and said (d) are repeated iteratively.

92. A method according to claim 88, comprising selecting a material property such that elongation of said material is consistent with the geometric design of said stent.

93. A stent manufactured according to specification generated by the method of claim 88.
